(19) United States
(12) Patent Application Publication
(10) Pub. No.: US 2010/0004333 A1
(43) Pub. Date: Jan. 7, 2010

(54) COMPOSITIONS AND METHODS FOR TREATING CARDIOVASCULAR DISORDERS

(76) Inventor: Roland O. Stocker, New South Wales (AU)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/722,053

(22) PCT Filed: Dec. 16, 2005
(86) PCT No.: PCT/AU2005/001904

§ 371 Date: Sep. 23, 2009

(30) Foreign Application Priority Data
Dec. 17, 2004 (AU) 2004907193
May 2, 2005 (AU) 2005902208

(51) Int. Cl. A61K 31/095; C12N 5/00; A61K 31/222; C07C 321/28; C07C 391/02; A61P 9/00

(52) U.S. Cl. 514/548; 435/375; 514/706; 560/194; 568/75; 568/717

(57) ABSTRACT

The present invention relates to compounds and methods for the treatment of cardiovascular diseases and disorders. Compounds according to the present invention may comprise an optionally substituted phenyl ring linked to an aromatic or alkyl group by a spacer, wherein the spacer comprises two groups selected from selenium, sulfur, S(O) and S(O)₂ and may further optionally comprise an alkylene, alkenylene, cycloalkylene or arylene moiety between the respective selenium, sulfur, S(O) and S(O)₂ groups.

### FIELD OF THE INVENTION

The present invention relates to compounds and methods for the treatment of cardiovascular diseases and disorders.

### BACKGROUND OF THE INVENTION

Heart disease can result from many factors relating to poor functioning of heart tissue which may manifest in commonly known conditions such as angina, stroke, or heart attack. The underlying mechanisms of heart disease are not completely understood. However, it is known that lipids, such as cholesterol, are actively involved and may contribute to atherosclerosis, i.e., the clogging of arteries, and the build-up of deposits that may eventually lead to heart disease, or stroke. According to the ‘oxidative modification theory’ of atherosclerosis, it is oxidised lipid, particularly in the form of oxidised low-density lipoprotein (LDL) particles that initiate and contribute to the subsequent development of atherogenesis.

Phenolic compounds are generally known to be good radical scavengers. In vitro, phenolic compounds effectively inhibit the peroxidation of lipids in homogeneous solution (e.g., when the lipids are dissolved in an organic solvent) that itself is a free radical process. For example, α-tocopherol (the most active form of vitamin E) is a phenolic compound that effectively inhibits radical-induced lipid peroxidation in vitro, and the vitamin is commonly thought to be the most important inhibitor of lipid oxidation in biological systems. Probucol (initially introduced as a lipid-lowering drug) is also a phenolic compound which exhibits radical scavenging activity and is thought to attenuate cardiovascular disease by preventing the oxidation of LDL. For this reason, the literature focuses on compounds having a phenolic residue for anti-atherogenic activity.

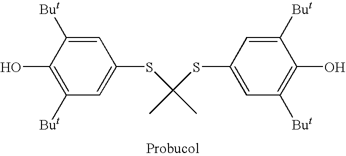

However, recent results show that the process of peroxidation of lipids in homogeneous solution is fundamentally different from the process of peroxidation of lipids in LDL particles (that represent an emulsion of ‘lipid droplets in aqueous solution’). For example, α-tocopherol does not necessarily inhibit, and in some circumstances can even promote, the oxidation of LDL lipids, and this may in part explain why vitamin E supplements have generally failed to provide protection against cardiovascular disease in recent controlled prospective studies in humans. Furthermore, recent results have also shown that inhibition of lipid peroxidation by phenolic compounds does not account for in vivo protective activity particularly as inhibition of lipoprotein lipid oxidation in the vessel wall and atherosclerosis are two events that can be dissociated from each other.

EP 1 464 639 (entitled “Succinic acid ester of probucol for the inhibition of the expression of VCAM-1”) discloses analogues of probucol having at least one phenolic residue as inhibitors of both, lipid oxidation and the expression of vascular cell adhesion molecule-1 (VCAM-1). EP 1 464 639 also disclosed the use of phenolic analogues of probucol for the treatment of diseases mediated by VCAM-1, including cardiovascular disorders.

However, irrespective of the contribution of LDL oxidation to the disease process, the presence alone of a phenolic residue cannot account for the protective activity of is probucol and the probucol analogues, as compounds such as compounds A and B below, do not have anti-atherosclerotic activity despite the presence of phenolic residues.

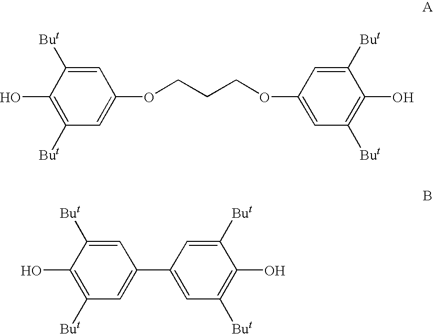

There is a need for alternative therapies for cardiovascular disorders, including treatment and prevention of atherosclerosis and restenosis.

### SUMMARY OF THE INVENTION

In a first aspect, the present invention relates to compounds of general Formula (I):

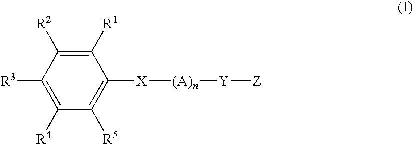

wherein

X is selected from S, Se, S(O) and S(O)₂;

Y is selected from S, Se, S(O) and S(O)₂;

A comprises one or more groups selected from optionally substituted C₁₋₆ alkylene, optionally substituted C₂₋₆ alkenylene; optionally substituted C₃₋₁₀ cycloalkylene; and optionally substituted arylene;

n is 0 or 1;

Z is selected from optionally substituted aryl and optionally substituted heteroaryl, optionally substituted alkyl, optionally substituted alkoxy, and NR¹³R¹⁴;

R¹, R², R³, R⁴, and R⁵ may be the same or different and are independently selected from the group consisting of hydrogen, halogen, hydroxyl, thiol, —NR¹³R¹⁴, nitro, cyano, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted aryl, optionally substituted aryl(C₁₋₆ alkyl), optionally substituted (C₁₋₆ alkyl)aryl, optionally substituted heteroaryl, optionally substituted C₃₋₁₀ heterocycloalkyl, C(O)R¹¹, OR¹², SR¹², CH₂OR¹², CH₂NR¹³R¹⁴, C(O)OR¹² and C(O)NR¹³R¹⁴;

R¹¹ is selected from OH, C₁₋₆ alkyl, and C₁₋₆ alkenyl;

R¹² is selected from the group consisting of hydrogen, optionally substituted C₁₋₁₀ to alkyl, optionally substituted C₂₋₁₀ alkenyl, optionally substituted C₂₋₁₀ alkynyl, optionally substituted C₃₋₁₀ cycloalkyl, optionally substituted aryl, —C(O)(C₁₋₆)alkyl-CO₂R¹⁵, —C(O)(C₂₋₆)alkenyl-CO₂R¹⁵, and —(O)NR¹³R¹⁴;

R¹³ and R¹⁴ may be the same or different and are individually selected from hydrogen, C₁₋₆ alkyl, C₂₋₆ alkenyl, C₂₋₆ alkynyl, C₃₋₆ cycloalkyl, C₃₋₆ heterocycloalkyl, aryl, (C₁₋₆)alkylaryl, and heteroaryl; and

R¹⁵ is H or C₁₋₄ alkyl;

and salts thereof.

In a second aspect the present invention relates to pharmaceutical compositions comprising at least one compound of Formula (I) as defined in the first aspect of the invention, together with pharmaceutically acceptable excipient, diluents and/or adjuvants.

In a third aspect the present invention relates to a method of treating a cardiovascular disorder in a vertebrate, said method comprising administering to said vertebrate an effective amount of a compound according to Formula (I) as defined in the first aspect of the invention or a composition according to the second aspect of the invention.

In a fourth aspect the invention relates to the use of a compound of Formula (I) according to the first aspect of the invention for the manufacture of a medicament for treating a cardiovascular disorder.

In a fifth aspect the invention relates to a process for preparing a pharmaceutical composition comprising homogeneously mixing a compound according to the first aspect of the invention with a pharmaceutically acceptable adjuvant, diluent and/or carrier.

### ABBREVIATIONS

AAPH—2,2′-azobis(2-amidino-propane)hydrochloride
ABI—aortic balloon injury
ACh—acetylcholine
apoE−/−, apolipoprotein E-deficient
apoE−/−; LDLr−/−, apolipoprotein E and LDL receptor-deficient
BA—balloon angioplasty
BP—3,3′,5,5′-tetra-tert-butyl-4,4′-bisphenol
C18:2—cholesteryl linoleate
C20:4—cholesteryl arachidonate
CE—cholesteryl esters (C18:2 plus C20:4)
CE-O(O)H—hydroxides and hydroperoxides of cholesteryl ester
CoQ₁₀—ubiquinone-10
cGMP—guanosine 3′,5′-cyclic monophosphate
DPQ—3,3′,5,5′-tetra-tert-butyl-4,4′-diphenoquinone
DTBP—4,4′-dithiobis(2,6-di-tert-butyl-phenol)
DTBP-s—DTBP monosuccinate
eNOS—endothelial nitric oxide synthase
HDL—high density lipoprotein
HPLC—high performance liquid chromatography
HO-1—heme oxygenase-1
HOCl—hypochlorous acid
LDL—low density lipoprotein
LOOH—lipid hydroperoxides of cholesteryl esters
O₂^{.-}—superoxide anion radical
P—probucol
PTCA—percutaneous transluminal coronary angioplasty
sGC—soluble guanylyl cyclase
STBP—2,6-di-t-butyl-4-(3,5-di-t-butyl-4-hydroxyphenylselanylthio) phenol
α-TOH—α-tocopherol
α-T3—α-tocotrienol
VCAM—vascular cell adhesion molecule

### BRIEF DESCRIPTION OF THE FIGURES

FIG. 1 Site-specific effect of probucol on atherosclerosis in apoE−/− mice. Lesion sizes at sinus, arch and thoracic/abdominal aorta after six months intervention in probucol-treated mice () expressed relative to the corresponding lesion size in control animals (◯). Results show mean±SEM for 17 mice per group and for each site. *Significantly different from corresponding control value (p<0.05).

FIG. 2 Site-specific effect of probucol on arterial accumulation of non-oxidized lipids and α-tocopherol. Lesion content of C (A), CB (B) and α-tocopherol (C) after six months intervention in probucol-treated mice (filled symbols) expressed relative to controls (open symbols). Results show mean±SEM of four independent pools containing 19 (control) and 15 (probucol) respective sections. *Significantly different from corresponding control (p<0.05).

FIG. 3 Site-specific effect of probucol on arterial lipid oxidation. Lesion content of is parent lipid-standardized CE-OOH (A), F₂-isoprostanes (B) and 7KC (C) after six months intervention in probucol-treated mice (filled symbols) relative to controls (open symbols). Results show mean±SEM and, for A and C, represent four independent pools containing 19 (control) and 15 (probucol) sections; for B, ten separate sections were used for control and probucol. *Significantly different from corresponding control (p<0.05).

FIG. 4 Macrophage and extra-cellular matrix content in the aortic sinus of atherosclerotic lesions of apoE−/− mice. Aortic sinus lesions of apoE−/− mice fed a high fat diet without (A, C, E) and with 1% (w/w) probucol (B, D, F) were stained for macrophages (A, B) or collagen (C—F) as described in the Methods section. Accumulation of macrophages/macrophage foam cells (brown staining) is evident along the luminal side of the lesions from control and probucol-treated mice. Bright field images (C, D) were used to determine lesion areas, whereas polarization microscopy (E, F) was used to analyze the collagen-containing area that exhibits strong birefringence (red staining). The sections shown are representative of the results seen in six different animals (for control and probucol). Calibration bar represents 2 μm.

FIG. 5 Site-specific metabolism of probucol in aortas of apoE−/− mice. Lesion content of probucol (A) and its proportion present as bisphenol and diphenoquinone (B) after six months intervention. Data in A is given in nmol per mg protein (◯) or mmol per mol C+CE (). Results show mean±SD from four independent pools each containing 15 respective sections. Where SD cannot be seen, they are smaller than the symbol size. *′^{†}Significantly different from corresponding sinus and arch value, respectively (p<0.05).

FIG. 6 Dietary probucol and DTBP but not BP attenuate HOCl-induced endothelial dysfunction. Aortic rings from rabbits fed normal diet (◯) or normal diet supplemented with 1% probucol (), 0.2% DTBP (▴) or 0.02% BP (♦) for 4 weeks were pre-constricted and relaxation to ACh assessed as described in the Methods Section. (A) Dose response to ACh with (◯, , ▴, ▪) and without (Δ) 5 min pre-incubation with 400 μM HOCl. (B) Tissue cGMP was measured in aortic rings from rabbits fed normal or supplemented diets that were pre-constricted and exposed to 1 μM ACh after 5 min pre-incubation with 400 μM HOCl. Tissue cGMP was then expressed relative to that in control rings in the absence of HOCl-treatment, with 100% corresponding to 454±46 pmol/g wet tissue. (C) Drug content in rings used for relaxation studies. Data show mean±SEM from rings of 6 animals per treatment. *Significantly different from control (P<0.05).

FIG. 7 In vitro added probucol and DTBP attenuate HOCl-induced endothelial dysfunction. (A) Aortic rings from rabbits fed normal diet were pre-incubated for 10 min without (□) or with 10 (♦, ⋄), 25 (▴, Δ) or 100 μM (,◯) probucol (filled symbols) or DTBP (open symbols), washed and then exposed to 400 μM HOCl prior to pre-constriction and relaxation in response to ACh. *P<0.05 for comparison of untreated rings versus rings treated with probucol or DTBP. (B) Tissue cGMP in aortic rings exposed to 400 μM HOCl for 5 min after pre-incubation in the absence and presence of 100 μM probucol or DTBP, and (C) aortic drug content before exposure of HOCl. cGMP was expressed relative to that in control rings as described in the Legend to FIG. 1, with 100% corresponding to 454±46 pmol/g wet tissue. Data show mean±SEM from rings of 6 animals per treatment. *Significantly different from control (P<0.05).

FIG. 8 Oxidation of probucol by HOCl. Probucol (final concentration 1 mM) dissolved in hexane was oxidized with increasing concentrations of reagent HOCl for 60 min at 37° C. (A) Consumption of probucol (◯) and formation of DTBP () and DPQ plus compounds 2, 4, and 6 (Δ) was monitored by HPLC as described in the Methods Section. (B) Representative chromatograms of reaction mixture before (top) and after oxidant exposure (bottom) monitored at 270 (solid line) and 420 nm (broken line). Eluting products were labelled sequentially 1-7, purified by semi-preparative HPLC and used retrospectively for quantification in panel (A). Compound 7, eluting at 29.2 min and absorbing at 420 nm was identified as DPQ using an authentic standard. Panels (C) and (D) show the corresponding results for oxidation of DTBP with HOCl. Results in (A) and (C) show mean±SEM for 3 separate experiments.

FIG. 9 Negative-ion electrospray mass spectra of the isolated products obtained from the reaction between probucol and HOCl. Probucol (1 mM in hexane, 2 mL) was incubated with 2 mM HOCl at 37° C. for 60 min, before addition of 1 mL water, removal of the hexane phase for analysis of the oxidation products by semi-preparative scale gradient reversed-phase HPLC. Compounds eluting sequentially (1 to 6 as per FIG. 8B) were isolated, and subsequently analysed with negative ion ESI-MS as described in the Methods section.

FIG. 10 Observed rate constants for the reaction of HOCl with probucol and DTBP. Reactions were performed in aqueous ethanol (70% EtOH, v/v) at 25° C. and followed for 500 s. (A) Representative spectral changes during oxidation of 50 μM probucol with 250 μM HOCl. The arrow indicates the increase in absorbance at 440 nm due to DPQ formation. (B) Time-dependent change in absorbance at 440 nm (solid line) during probucol oxidation indicating bi-phasic nature of the reaction that was simulated to best fit (dashed line) yielding a low residual (inset). (C) Plots of observed rate constants (k_{obs}) versus HOCl concentration for each phase of probucol (filled circle and square) or DTBP (hatched square) oxidation, fitted by linear regression to yield the apparent rate constants k₁ and k₂ for probucol and k for DTBP. Data show mean±SD of 3 separate experiments. Where error bars are not seen the symbol is larger. Note the overlap of filled and hatched squares.

FIG. 11 Aortic content of probucol and its metabolites before and after HOCl exposure. Aortas from rabbits fed normal diet supplemented with (A) 1% probucol or (C) 0.2% DTBP for 4 weeks were analyzed for probucol and its metabolites before (open bars) and after exposure to 400 μM HOCl (filled bars) as described in the Methods Section. Similarly, aortic segments obtained from control rabbits were supplemented in vitro with (B) 100 μM probucol or (D) DTBP and analyzed for probucol and its metabolites before (open bars) and after exposure to 400 μM HOCl (filled bars). Data show mean±SEM of 3 separate experiments using aortic rings from three different animals. *Significantly different from corresponding vessel without HOCl-treatment (P<0.05).

FIG. 12 DTBP, not BP, inhibits atherosclerosis in apoE−/− mice similar to probucol. a, Representative cross sections of abdominal aorta from control (Ctrl) and three treatment groups stained for macrophages, indicating respective lesion size (×400). b, Site-specific effect of probucol (filled diamond), DTBP (filled squared) and BP (filled triangle) on atherosclerosis as compared to control (circle), n=10 for each site (each using 2, 2, 6 and 3 sections for aortic sinus, arch, thoracic and abdominal aorta, respectively). c, Plasma cholesterol, n=10. d and e, Total neutral lipids (NL, comprised of cholesterylesters and triglycerides) and their hydroperoxides (LOOH) standardized to NL, n=3 pools of 5 aortas per pool. f and g, Average lesion area covered by Mac-3⁺-cells (i.e., macrophages) and PCNA⁺-(i.e., proliferating) cells in arch, thoracic and abdominal aorta, as compared to control, n=3 for each site (3 sections/site). h, FPLC chromatograms of lipoproteins from pooled plasma (n=10) of control, probucol, DTBP and BP animals. *, P<0.05 compared to control.

FIG. 13 DTBP, not BP, inhibits intimal hyperplasia in rabbits in response to vessel injury similar to probucol. a, Representative Verhoeff's hematoxylin-stained cross sections (×10). b, I/M ratios of vessels from control and drug-treated rabbits after 6 weeks of ABI (8 serial sections per aortic segment, 100 μm apart). c and d, Total neutral lipids (NL) and their hydroperoxides (LOOH). e, Time-dependent changes in plasma cholesterol, with symbols as described in Legend to FIG. 12. All results are from 6 is rabbits per group. *, P<0.05 compared to control.

FIG. 14 DTBP concentration-dependently inhibits intimal hyperplasia in rabbits in response to vessel injury. I/M ratios of vessels from control (n=6) and drug-treated rabbits (n=6 for each drug concentration) after 6 weeks of ABI (8 serial sections per aortic segment, 100 μm apart).

FIG. 15 Probucol and DTBP, not BP, promote functional re-endothelialization in rabbits after injury. a, Representative longitudinal section with branch orifice (×20) showing denuded and CD-31⁺ re-endothelialized aortic surface (red arrows) distal and proximal to the branch orifice, respectively (×400), after 6 weeks of injury. b, Re-endothelialization assessed by the length (mm) of section covered by CD-31⁺ cells from branch orifice for the groups after 6 weeks of injury (3-6 serial sections per segment, 100 μm apart). c and e, Relaxation to acetylcholine (ACh) and sodium nitroprusside (SNP) of pre-constricted aortic ring taken from rabbits of the four groups. Symbols are as described in Legend to FIG. 2. d, Cyclic GMP content of aortic rings exposed to ACh after pre-incubation in isobutylmethylxanthine. All results are from 6 rabbits per group. *, P<0.05 compared to control.

FIG. 16 Probucol and DTBP, not BP, induce heme oxygenase. a, Representative cross sections taken from control and treated rabbits 4 days after ABI, and stained for heme oxygenase-1 (HO-1) (×400), n=4 for each treatment (3 sections/aorta). b and c, HO-1 mRNA and HO activity in aortas taken from control and treated rabbits 4 days after ABI, n=8 per treatment d and e, Apoptosis indicated by TUNEL⁺-cells and proliferation indicated by PCNA⁺-cells 4 (open bars) and 42 days (closed bars) after ABI, n=4-6 for each treatment (3 sections/aorta). *, P<0.05 compared to control.

FIG. 17 Oxidation of the sulfur atoms of probucol to the disulfoxide is a proposed first step in HOCl-mediated conversion of probucol to DTBP. This proposed mechanism is distinct from the oxidation of the phenolic groups of probucol.

FIG. 18 Proposed non-radical mechanism for oxidation of probucol.

FIG. 19 Heme oxygenase-1 (HO-1) is a target for probucol and DTBP. Blocking heme oxygenase activity via administration of tin protoporphyrin prevented the ability of probucol and DTBP to inhibit intimal thickening in response to arterial balloon injury (FIG. 19a), to promote re-endothelialization (FIG. 19b), and to inhibit vascular smooth muscle cell proliferation (FIG. 19c).

FIG. 20 Vitamin E fails to inhibit intimal hyperplasia and does not promote re-endothelialization or induce HO-1. (A) HO-1 mRNA assessed by real time RT-PCR in rabbit aortic smooth muscle cells cultured for 24 hours in the presence of vehicle is (control), probucol (50 μM) or α-tocopherol (vitamin E, 50 μM). (B) Re-endothelialization assessed by Evans blue staining for the three groups of rabbits in (A) 3 weeks after injury. (C) Intima-to-media ratio of vessels from control rabbits and animals treated with probucol or α-tocopheryl acetate (vitamin E) (n=6 per group) 3 weeks after aortic balloon injury (5 serial sections per aortic segment, 100 μm apart). Results show mean±SEM of a triplicate experiment performed twice with similar results obtained in both experiments. *, P<0.05 compared to control; **, P<0.01 compared to control.

FIG. 21 DTBP and its analogs inhibit intimal hyperplasia in response to vessel injury: 48 New Zealand White rabbits (1.8-2.2 kg) were fed normal chow (100 g/day) without (Ctrl, n=12) or with DTBP (0.02% wt/wt, n=6), STBP (0.02% wt/wt, n=7) or DTBP-s (0.02%, n=11; or 0.1% wt/wt, n=7) for 9 weeks. Aortic balloon-injury was carried out at the end of week three. *, P<0.01 versus Ctrl; ^{#}, P<0.05 versus 0.02% DTBP-s.

### DEFINITIONS

The following are some definitions that may be helpful in understanding the description of the present invention. These are intended as general definitions and should in no way limit the scope of the present invention to those terms alone, but are put forth for a better understanding of the following description.

Unless the context requires otherwise or specifically stated to the contrary, integers, steps, or elements of the invention recited herein as singular integers, steps or elements clearly encompass both singular and plural forms of the recited integers, steps or elements.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

As used herein, the term “alkyl group” includes within its meaning monovalent (“alkyl”) and divalent (“alkylene”) straight chain or branched chain saturated aliphatic groups having from 1 to 10 carbon atoms, eg, 1, 2, 3, 4, 5, 6, 7, 8, 9, or 10 carbon atoms. For example, the term alkyl includes, but is not limited to, methyl, ethyl, 1-propyl, isopropyl, 1-butyl, 2-butyl, isobutyl, tert-butyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl, and the like.

The term “alkenyl group” includes within its meaning monovalent (“alkenyl”) and divalent (“alkenylene”) straight or branched chain unsaturated aliphatic hydrocarbon groups having from 2 to 10 carbon atoms, eg, 2, 3, 4, 5, 6, 7, 8, 9, or 10 carbon atoms. Examples of alkenyl groups include but are not limited to ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 1-nonenyl, 1-decenyl, and the like.

The term “alkynyl group” as used herein includes within its meaning monovalent (“alkynyl”) and divalent (“alkynylene”) straight or branched chain unsaturated aliphatic hydrocarbon groups having from 2 to 10 carbon atoms and having at least one triple bond. Examples of alkynyl groups include but are not limited to ethynyl, 1-propynyl, 1-butynyl, 2-butynyl, 1-methyl-2-butynyl, 3-methyl-1-butynyl, 1-pentynyl, 1-hexynyl, methylpentynyl, 1-heptynyl, 2-heptynyl, 1-octynyl, 2-octynyl, 1-nonyl, 1-decynyl, and the like.

The term “cycloalkyl” as used herein refers to cyclic saturated aliphatic groups and includes within its meaning monovalent (“cycloalkyl”), and divalent (“cycloalkylene”), saturated, monocyclic, bicyclic, polycyclic or fused polycyclic hydrocarbon radicals having from 3 to 10 carbon atoms, eg, 3, 4, 5, 6, 7, 8, 9, or 10 carbon atoms. Examples of cycloalkyl groups include but are not limited to cyclopropyl, 2-methylcyclopropyl, cyclobutyl, cyclopentyl, 2-methylcyclopentyl, 3-methylcyclopentyl, cyclohexyl, and the like.

The term “cycloalkenyl” as used herein, refers to cyclic unsaturated aliphatic groups and includes within its meaning monovalent (“cycloalkenyl”) and divalent (“cycloalkenylene”), monocyclic, bicyclic, polycyclic or fused polycyclic hydrocarbon radicals having from 3 to 10 carbon atoms and having at least one double bond, of either E, Z, cis or trans stereochemistry where applicable, anywhere in the alkyl chain. Examples of cycloalkenyl groups include but are not limited to cyclopropenyl, cyclopentenyl, cyclohexenyl, and the like.

The term “heterocycloalkyl” as used herein, includes within its meaning monovalent (“heterocycloalkyl”) and divalent (“heterocycloalkylene”), saturated, monocyclic, bicyclic, polycyclic or fused hydrocarbon radicals having from 3 to 10 ring atoms wherein 1 to 5 ring atoms are heteroatoms selected from O, N, NH, or S. Examples include pyrrolidinyl, piperidinyl, quinuclidinyl, azetidinyl, morpholinyl, tetrahydrothiophenyl, tetrahydrofuranyl, tetrahydropyranyl, and the like.

The term “heterocycloalkenyl” as used herein, includes within its meaning monovalent (“heterocycloalkenyl”) and divalent (“heterocycloalkenylene”), saturated, monocyclic, bicyclic, polycyclic or fused polycyclic hydrocarbon radicals having from 3 to 10 ring atoms and having at least 1 double bond, wherein from 1 to 5 ring atoms are heteroatoms selected from O, N, NH or S.

The term “heteroaromatic group” and variants such as “heteroaryl” or “heteroarylene” as used herein, includes within its meaning monovalent (“heteroaryl”) and divalent (“heteroarylene”), single, polynuclear, conjugated and fused aromatic radicals having 6 to 20 atoms wherein 1 to 6 atoms are heteroatoms selected from O, N, NH and S. Examples of such groups include pyridyl, 2,2′-bipyridyl, phenanthrolinyl, quinolinyl, thiophenyl, and the like.

The term “halogen” or variants such as “halide” or “halo” as used herein refers to fluorine, chlorine, bromine and iodine.

The term “heteroatom” or variants such as “hetero-” as used herein refers to O, N, NH and S.

The term “alkoxy” as used herein refers to straight chain or branched alkyloxy groups. Examples include methoxy, ethoxy, n-propoxy, isopropoxy, sec-butoxy, tert-butoxy, and the like.

The term “amino” as used herein refers to groups of the form —NRₐR_{b} wherein Rₐ and R_{b} are individually selected from the group including but not limited to hydrogen, optionally substituted alkyl, optionally substituted alkenyl, optionally substituted alkynyl, and optionally substituted aryl groups.

The term “aromatic group”, or variants such as “aryl” or “arylene” as used herein refers to monovalent (“aryl”) and divalent (“arylene”) single, polynuclear, conjugated and fused residues of aromatic hydrocarbons having from 6 to 10 carbon atoms. Examples of such groups include phenyl, biphenyl, naphthyl, phenanthrenyl, and the like.

The term “aralkyl” as used herein, includes within its meaning monovalent (“aryl”) and divalent (“arylene”), single, polynuclear, conjugated and fused aromatic hydrocarbon radicals attached to divalent, saturated, straight and branched chain alkylene radicals.

The term “heteroaralkyl” as used herein, includes within its meaning monovalent (“heteroaryl”) and divalent (“heteroarylene”), single, polynuclear, conjugated and fused aromatic hydrocarbon radicals attached to divalent saturated, straight and branched chain alkylene radicals.

The term “optionally substituted” as used herein means the group to which this term refers may be unsubstituted, or may be substituted with one or more groups independently selected from alkyl, alkenyl, alkynyl, thioalkyl, cycloalkyl, cycloalkenyl, heterocycloalkyl, halo, carboxyl, haloalkyl, haloalkynyl, hydroxyl, alkoxy, thioalkoxy, alkenyloxy, haloalkoxy, haloalkenyloxy, nitro, amino, nitroalkyl, nitroalkenyl, nitroalkynyl, nitroheterocyclyl, alkylamino, dialkylamino, alkenylamine, alkynylamino, acyl, alkenoyl, alkynoyl, acylamino, diacylamino, acyloxy, alkylsulfonyloxy, heterocycloxy, heterocycloamino, haloheterocycloalkyl, alkylsulfenyl, alkylcarbonyloxy, alkylthio, acylthio, phosphorus-containing groups such as phosphono and phosphinyl, aryl, heteroaryl, alkylaryl, alkylheteroaryl, cyano, cyanate, isocyanate, —C(O)NH(alkyl), and —C(O)N(alkyl)₂.

In the context of this invention the term “co-antioxidant” refers to inhibitors of lipoprotein lipid oxidation that are effective in blood vessel walls in vivo. A detailed description of the characterization of co-antioxidants is given in Journal American Chemical Society 1993, 115:6029-6044; Proceedings of the National Academy of Sciences USA 1993, 90:45-49; and Journal of Biological Chemistry 1995, 270:5756-5763 which are incorporated herein by reference. Co-antioxidants differ from classic “antioxidants” or “radical scavengers” in that the former prevent the pro-oxidant activity of α-tocopherol in the peroxidation of lipoprotein lipids by inhibiting the process of tocopherol-mediated peroxidation. Inhibition of tocopherol-mediated peroxidation by a co-antioxidant is achieved via the combination of (i) reducing α-tocopheroxyl radical to α-tocopherol and (ii) aiding the transfer of the radical character from the lipoprotein particle into the surrounding aqueous environment such that reformation of α-tocopheroxyl radical is prevented. Co-antioxidants may be routinely identified by in vivo analysis of the effects of the inhibitors in blood vessel walls using a suitable animal model such as Watanabe Heritable Hyperlipidemic (WHHL) rabbits, apoE−/− mice, or cholesterol-fed balloon-injured New Zealand White rabbits. Alternatively, co-antioxidants may be identified through in vitro assays which are capable of demonstrating such efficacy, such as for example assays described in J Lipid Research 1996, 37:853-867 which is incorporated herein by reference.

In the context of this specification, the term “vessels” includes all fluid or air filled vessels of the body which are lined with endothelium, including for example, blood vessels, such as arteries.

In the context of this specification the term “functional endothelium” refers to blood vessel containing endothelial cells that to suitable agonists by the production of nitric oxide that itself acts on the underlying smooth muscle cells by activating soluble guanylyl cyclase with resultant formation of cyclic guanosine monophosphate (cGMP) and relaxation of the blood vessel.

In the context of this specification the term “administering” and variations of that term including “administer” and “administration”, includes contacting, applying, delivering or providing a compound or composition of the invention to an organism, or a surface by any appropriate means.

In the context of this specification, the term “vertebrate” includes humans and individuals of any species of social, economic or research importance including but not limited to members of the genus ovine, bovine, equine, porcine, feline, canine, primates (including human and non-human primates), rodents, murine, caprine, leporine, and avian.

In the context of this specification, the term “treatment”, refers to any and all uses which remedy a disease state or symptoms, prevent the establishment of disease, or otherwise prevent, hinder, retard, or reverse the progression of disease or other undesirable symptoms in any way whatsoever.

In the context of this specification the terms “therapeutically effective amount” and “diagnostically effective amount”, include within their meaning a sufficient but non-toxic amount of a compound or composition of the invention to provide the desired therapeutic or diagnostic effect. The exact amount required will vary from subject to subject depending on factors such as the species being treated, the age and general condition of the subject, the severity of the condition being treated, the particular agent being administered, the mode of administration, and so forth. Thus, it is not possible to specify an exact “effective amount”. However, for any given case, an appropriate “effective amount” may be determined by one of ordinary skill in the art using only routine experimentation.

### DESCRIPTION OF PREFERRED EMBODIMENTS OF THE INVENTION

The present invention relates to compounds, compositions and methods for treating cardiovascular disorders.

In one aspect, the present invention relates to compounds of general Formula (I):

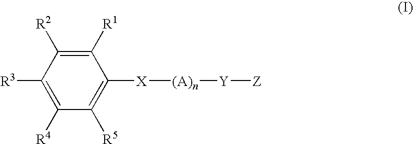

wherein

X is selected from S, Se, S(O) and S(O)₂;

Y is selected from S, Se, S(O) and S(O)₂;

A comprises one or more groups selected from optionally substituted C₁₋₆ alkylene, optionally substituted C₂₋₆ alkenylene; optionally substituted C₃₋₁₀ cycloalkylene; and optionally substituted arylene;

n is 0 or 1;

Z is selected from optionally substituted aryl and optionally substituted heteroaryl, optionally substituted alkyl, optionally substituted alkoxy, and NR¹³R¹⁴;

R¹, R², R³, R⁴, and R⁵ may be the same or different and are independently selected from the group consisting of hydrogen, halogen, hydroxyl, thiol, —NR¹³R¹⁴, nitro, cyano, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted aryl, optionally substituted aryl(C₁₋₆ alkyl), optionally substituted (C₁₋₆ alkyl)aryl, optionally substituted heteroaryl, optionally substituted C₃₋₁₀ heterocycloalkyl, C(O)R¹¹, OR¹², SR¹², CH₂OR¹², CH₂NR¹³R¹⁴, C(O)OR¹² and C(O)NR¹³R¹⁴;

R¹¹ is selected from OH, C₁₋₆ alkyl, and C₁₋₆ alkenyl;

R¹² is selected from the group consisting of hydrogen, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted aryl, —C(O)(C₁₋₆)alkyl-CO₂R¹⁵, —C(O)(C₂₋₆)alkenyl-CO₂R¹⁵, and —(O)NR¹³R¹⁴;

R¹³ and R¹⁴ may be the same or different and are individually selected from hydrogen, C₁₋₆ alkyl, C₂₋₆ alkenyl, C₂₋₆ alkynyl, C₃₋₆ cycloalkyl, C₃₋₆ heterocycloalkyl, aryl, (C₁₋₆)alkylaryl, and heteroaryl; and

R¹⁵ is H or C₁₋₄ alkyl;

and salts thereof.

In one embodiment when n is 1, the spacer group “A” is present. In another embodiment when n is 0, the spacer group “A” is absent.

In one embodiment the compound is a compound of Formula (Ia):

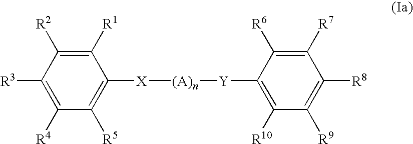

wherein

X is S or Se;

Y is S or Se;

A comprises one or more groups selected from optionally substituted C₁₋₆ alkylene, optionally substituted C₂₋₆ alkenylene; and optionally substituted C₃₋₁₀ cycloalkylene;

n is 0 or 1;

R¹, R², R³, R⁴, R⁵, R⁶, R⁷, R⁸, R⁹ and R¹⁰ may be the same or different and are independently selected from the group consisting of hydrogen, halogen, hydroxyl, thiol, —NR¹¹R¹², nitro, cyano, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted aryl, optionally substituted aryl(C₁₋₆ alkyl), optionally substituted (C₁₋₆ alkyl)aryl, optionally substituted heteroaryl, optionally substituted C₃₋₁₀ heterocycloalkyl, C(O)R¹¹, OR¹², CH₂OR¹², CH₂NR¹³R¹⁴, C(O)OR¹² and C(O)NR¹³R¹⁴;

R¹¹ is selected from OH, C₁₋₆ alkyl, and C₁₋₆ alkenyl;

R¹² is selected from the group consisting of hydrogen, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted aryl, —C(O)(C₁₋₆)alkyl-CO₂R¹⁵, —C(O)(C₂₋₆)alkenyl-CO₂R¹⁵, and C(O)NR¹³R¹⁴;

R¹³ and R¹⁴ may be the same or different and are individually selected from hydrogen, C₁₋₆ alkyl, C₂₋₆ alkenyl, C₂₋₆ alkynyl, C₃₋₆ cycloalkyl, C₃₋₆ heterocycloalkyl, aryl, (C₁₋₆)alkylaryl, and heteroaryl; and

R¹⁵ is H or C₁₋₄ alkyl;

and salts thereof.

In one embodiment the compound is a compound of Formula (Ib):

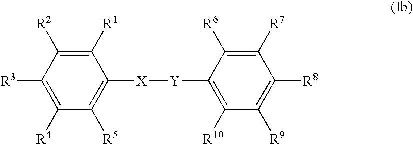

wherein

X is selected from S, Se, S(O) and S(O)₂;

Y is selected from S, Se, S(O) and S(O)₂; and

R¹-R¹⁰ are as defined for Formula (Ia).

With reference to Formulae (I), (Ia) and (Ib), in one embodiment, X is S and Y is Se. In another embodiment, X is Se and Y is S. In a further embodiment, X is Se and Y is Se.

In one embodiment, the optional substituents are independently selected from OH, SH, halogen, C₁₋₄ alkyl, C₁₋₄ alkenyl, O—(C₁₋₄ alkyl), S—(C₁₋₄ alkyl), cyano, amino, CO₂H and C(O)—O(C₁₋₆)alkyl.

R³ and R⁸ may be the same or different. In one embodiment, R³ and R⁸ are independently selected from hydroxyl, thiol, —NR¹³R¹⁴, cyano, C₁₋₆ alkyl, C₂₋₆ alkenyl, OR¹², C(O)OR¹² and C(O)NR¹³R¹⁴, wherein R¹², R¹³ and R¹⁴ are as defined above for Formula (I).

In another embodiment, R₃ and R₈ are independently selected from hydroxyl, O-malonate, O-succinate, O-glutarate, O-adipate, O-maleate and O-fumarate.

In one embodiment of the invention, the compound has the formula:

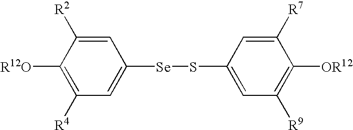

wherein

each R¹² is independently selected from hydrogen, C₁₋₁₀ alkyl and —C(O)(C₁₋₆)alkyl-CO₂R¹⁵;

R¹⁵ is selected from hydrogen and C₁₋₆ alkyl; and

R², R⁴, R⁷ and R⁹ are independently selected from methyl, ethyl, propyl, isopropyl, butyl, 1-methylpropyl, 2-methylbutyl, tert-butyl, pentyl, 2-methylpentyl, 3-methylpentyl and hexyl.

In one embodiment the compound is selected from:

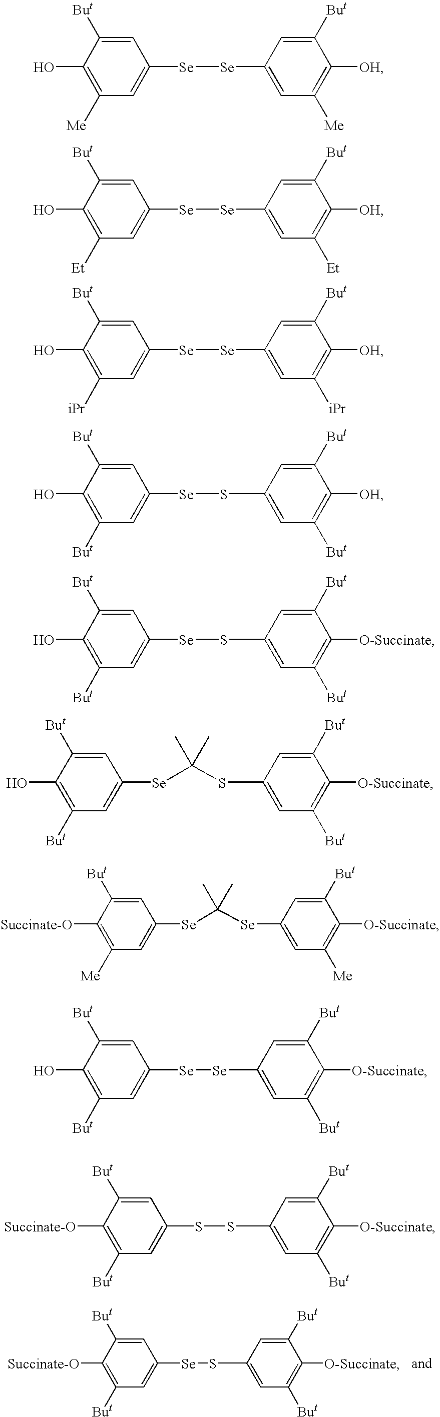

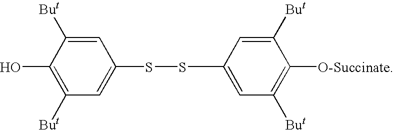

In various embodiments of the invention, compounds according to the present invention may comprise an optionally substituted phenyl ring linked to an aromatic or alkyl group by a spacer. In some embodiments, compounds according to the present invention comprise an optionally substituted phenyl ring linked to an aromatic group by a spacer.

The spacer comprises two groups selected from selenium, sulfur, S(O) and S(O)₂. The spacer may further optionally comprise an alkylene, alkenylene, cycloalkylene or arylene moiety between the respective selenium, sulfur, S(O) and S(O)₂ groups. Thus, for example, the spacer may comprise two adjacent selenium groups, a selenium group adjacent a sulfur group, a selenium group adjacent an S(O) group, and the like. Alternatively, respective selenium, sulfur, S(O) and S(O)₂ groups may be linked, for example, via a linear or branched carbon chain.

The terminal aromatic residues of compounds of Formula (I), i.e, the optionally substituted phenyl ring and “Z” group, may be the same or different. The aromatic residues may be substituted or unsubstituted. In one embodiment, the aromatic residues are each an optionally substituted phenyl ring. The aromatic residues may be substituted with one or more hydroxyl group(s). The aromatic residues may be substituted, respectively, with 1, 2, 3 or 4 alkyl group(s), wherein the alkyl group(s) may be the same or different. In some embodiments one or more hydroxyl group(s) may be functionalised, eg, as an ether, ester, or carbamate group.

Without intending to be limited to any particular mode of action, compounds of Formula (I) may undergo intra-cellular reduction to a substituted or unsubstituted thioaryl compound, or a substituted or unsubstituted selenoaryl compound. For example, compounds of Formula (I) may undergo intra-cellular reduction to substituted or unsubstituted mercaptophenol, or a substituted or unsubstituted selenophenol compound.

Compounds of Formula (I) may be prepared by methods known to those skilled in the art. Suitable methods are generally described, for example, and intermediates thereof are described, for example, in Houben-Weyl, Methoden der Organischen Chemie; J. March, Advanced Organic Chemistry, 4^{th} Edition (John Wiley & Sons, New York, 1992); D. C. Liotta and M. Volmer, eds, Organic Syntheses Reaction Guide (John Wiley & Sons, Inc., New York, 1991); R. C. Larock, Comprehensive Organic Transformations (VCH, New York, 1989), H. O. House, Modern Synthetic Reactions 2^{nd} Edition (W. A. Benjamin, Inc., Menlo Park, 1972).

Examples of general synthetic schemes for preparing compounds of Formula (I) are shown below:

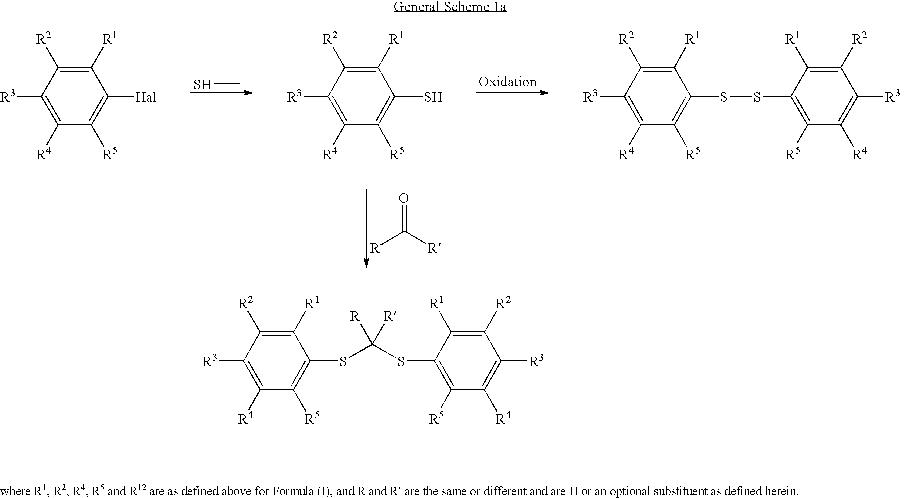

An alternative synthesis for thiophenol compounds involves formation of a disulfide compound (as described for example in Pastor et al., J Org Chem 1984; 49:5260-5262), followed by reduction to the corresponding thiol. The general synthesis may be described as follows:

Phenol+S₂Cl₂→Phenol-S—S-Phenol [1]

Phenol-S—S-Phenol+Zn/H⁺→Thiophenol [2]

Selenophenol compounds may be prepared according to the method described in Justus Liebigs Annalen der Chemie 1962; 657:5-12. The general synthesis is illustrated in General Scheme 1b:

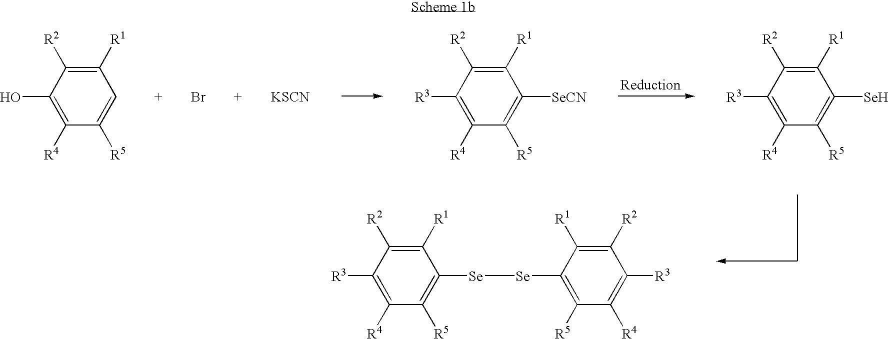

Due to the sensitivity of selenophenol compounds to oxidation, salts, for example the Zn salt, may be prepared.

An alternative strategy for preparing compounds of Formula (I) is shown in general Scheme 2:

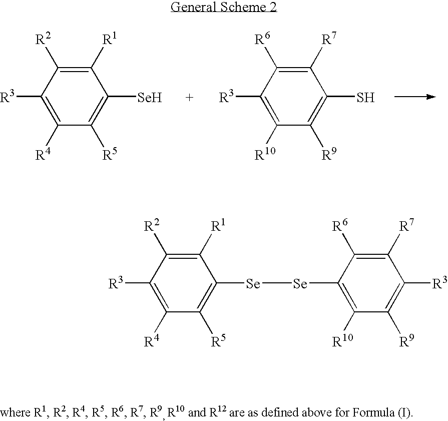

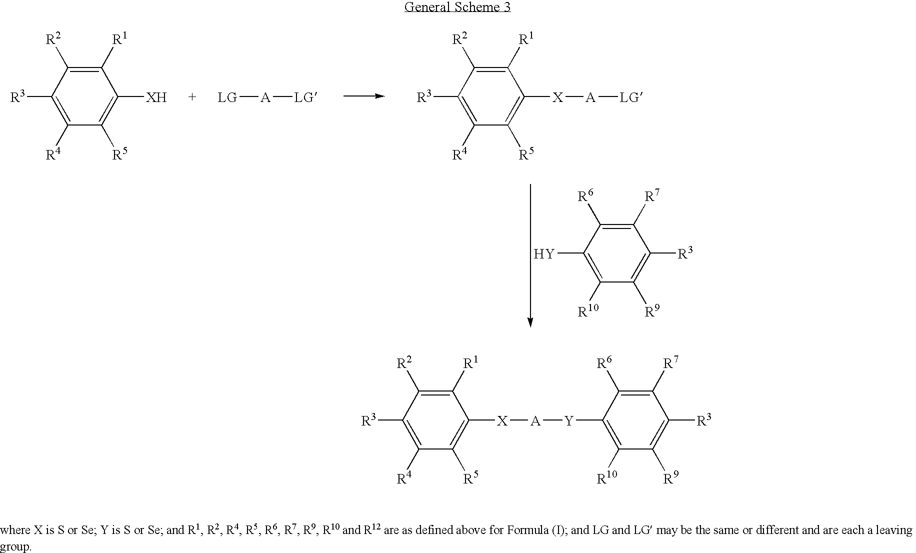

With reference to Scheme 3, suitable leaving groups are known to those skilled in the art and include, but are not limited to, halides, mesylate, tosylate, triflate, etc.

An alternative strategy for preparing compounds of Formula (I) is shown in general Scheme 4:

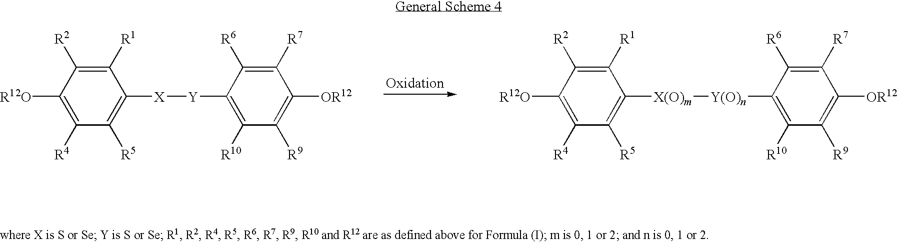

Those skilled in the art will also appreciate that various protecting groups may be used throughout a synthesis. Examples of protecting groups are known to those skilled in the art and have been described, for example, in Greene et al., Protective Groups in Organic Synthesis; John Wiley & Sons, 2^{nd} Edition, 1991. Those skilled in the art will also realise that compounds of Formula (I) may be prepared as salts and may comprise one or more of any suitable counterion. The counterion may be anionic, dianionic or polyanionic as appropriate. Where more than one counterion is present, the counterions may be the same or different. Examples of counterions include, but are not limited to halides (such as Cl⁻, Br⁻, I⁻), carboxylates, citrate, acetate, succinate, CF₃CO₂⁻, tosylate, nitrate, BF₄⁻, PF₆⁻, and OH⁻. The counterion(s) may be varied using techniques known to those skilled in the art, including for example, ion exchange and crystallisation.

The present invention includes within its scope all isomeric forms of the compounds disclosed herein, including all diastereomeric isomers, racemates and enantiomers. Thus, Formula (I) should be understood to include, for example, E, Z, cis, trans, (R), (S), (L), (D), (+), and/or (−) forms of the compounds, as appropriate in each case.

### Therapy

Compounds in accordance with the present invention may have in vivo activity associated with one or more of promotion of re-endothelialization, inhibition of smooth muscle cell proliferation, anti-inflammatory activity such as the inhibition of accumulation of pro-inflammatory cells in the affected vessel wall, and induction of heme oxygenase-1.

In one embodiment of the invention heme oxygenase is a target of compounds of formula (I). The heme oxygenase target may be heme oxygenase-1 (HO-1).

Accordingly, another aspect the present invention relates to a method of treating a cardiovascular disorder in a vertebrate, said method comprising administering to said vertebrate an effective amount of a compound according to Formula (I) as defined herein.

Another aspect of the invention relates to a compound of Formula (I) when used for the treatment of a cardiovascular disorder in a vertebrate.

In one embodiment, the vertebrate is a human.

A further aspect of the invention relates to the use of a compound of Formula (I) for the manufacture of a medicament for treating a cardiovascular disorder.

The cardiovascular disorder may be atherosclerosis. The cardiovascular disorder may be restenosis.

In accordance with an embodiment of the invention, a compound of Formula (I) may be administered together with a co-antioxidant.

Pre-treatment with one or more compounds of Formula (I) may be performed. For example, one or more compounds of Formula (I) may be administered 1, 2, 3, 4, 5, 6 or 7 days prior to an intervention, for example, prior to treating restenosis. Compounds of Formula (I) may be administered prior to or after angioplasty, PTCA or BA. Compounds of Formula (I) may be administered after denudation (removal of endothelial cells). Alternatively, compounds of Formula (I) may be administered prior to (e.g., 1, 2, 3, 4, 5, 6 or 7 days prior to) a denudation event, for example, prior to balloon angioplasty.

### Atherosclerosis

Atherosclerosis, i.e., the clogging of arteries, is characterized by the accumulation of cholesterol deposits in macrophages in large- and medium-sized arteries. This deposition leads to a proliferation of certain cell types within the arterial wall that gradually impinge upon the vessel lumen and impede blood flow. This process may be quite insidious lasting for decades until an atherosclerotic lesion, through physical forces from blood flow, becomes disrupted and deep arterial wall components are exposed to flowing blood, leading to thrombosis and compromised oxygen supply to target organs such as the heart and brain. The loss of heart and brain function as a result of reduced blood flow is termed heart attack and stroke, respectively, and these two clinical manifestations of atherosclerosis are often referred to as coronary artery disease and cerebrovascular disease. Coronary artery disease and cerebrovascular disease are commonly referred to by the collective term, cardiovascular disease.

With respect to the underlying pathology of atherosclerosis, there are a number of environmental and genetic “cardiovascular risk” factors that have proven predictive of the incidence of cardiovascular disease. Traits that are strongly and consistently associated with cardiovascular disease in a manner independent of other traits include age, gender, smoking, obesity, hypertension, diabetes mellitus, and serum cholesterol.

The association between low-density lipoprotein (LDL) cholesterol and atherosclerosis has been established based, in part, upon an experiment of nature. Familial hypercholesterolemia is an autosomal dominant disorder that affects approximately one in 500 persons from the general population. Heterozygotes for this disease manifest a two- to five-fold elevation in plasma LDL cholesterol that is due to a functional impairment of the LDL receptor, resulting in a defect in LDL clearance. Homozygotes for this disorder demonstrate a four- to six-fold elevation in plasma cholesterol that produces precocious atherosclerosis. In heterozygotes, 85% of individuals have experienced a myocardial infarction by the age of 60, and this age is reduced to 15 years inpatients homozygous for the disease. In the general population, the cardiovascular disease risk from increased LDL cholesterol is supported by observations that cholesterol-lowering therapy greatly diminishes the clinical manifestations of atherosclerosis, particularly since the advent of inhibitors of 3-hydroxy-3-methylglutaryl coenzyme A reductase (i.e., statins) that profoundly lower LDL cholesterol.

In contrast to the situation with LDL cholesterol, the relation between HDL cholesterol and atherosclerosis is an inverse one. The causal nature of this association is also supported by an experiment of nature—Tangier Disease. This autosomal co-dominant condition is characterized by the essential absence of HDL cholesterol levels due to a defect in the ATP binding cassette transporter-1 that impairs cholesterol efflux from cells. Tangier patients demonstrate a tissue cholesterol-loading syndrome, characterized by large tonsils, neuropathy, and premature coronary artery disease in some kindreds. Thus, considerable evidence supports the inverse relation between coronary artery disease and serum levels of HDL cholesterol.

Atherosclerosis manifests itself histological as arterial lesions known as plaques that have been extensively characterized into 6 major types of lesions that reflect the early, developing, and mature stages of the disease. In lesion-prone arterial sites, adaptive thickening of the intima is among the earliest histological changes. As macrophages accumulate lipid, type II lesions form as nodular areas of lipid deposition that are also known as “fatty streaks” and these represent lipid-filled macrophages (i.e., foam cells). Continued foam cell formation and macrophage necrosis can produce type III lesions that contain small extra-cellular pools of lipid. Types II and III lesions are readily apparent through the use of fat-soluble dyes that stain cholesterol esters accumulated in macrophages and the extra-cellular space. These early lesions are often evident by age 10 and can occupy as much as ⅓ of the aortic surface by the third decade. Developing lesions represent the next two types of lesions and are characterized by significant areas of extra-cellular lipid that represents the “core” of the atherosclerotic lesion. Type IV lesions are defined by a relatively thin tissue separation of the lipid core from the arterial lumen, whereas type V lesions exhibit fibrous thickening of this structure, also known as the lesion “cap.” These type IV and V lesions can be found initially in areas of the coronary arteries, abdominal aorta, and some aspects of the carotid arteries in the third to fourth decade of life.

Mature type VI lesions exhibit architecture that is more complicated and characterized by calcified fibrous areas with visible ulceration. These types of lesions, commonly referred to as atherosclerotic plaques, are often associated with clinical symptoms or arterial embolization. It was once thought that end-organ damage and infarction was due to gradual advancement of these lesions, but it is now known that the processes involved in precipitating heart attack and stroke are considerably more complex. Plaques contain a central lipid core that is most often hypo-cellular and may even include crystals of cholesterol that have formed in the aftermath of necrotic foam cells. In this late stage of lesion development, residual foam cells may be difficult to see, but have often left the core with an abundant quantity of tissue factor, an important activator of the clotting cascade. This lipid core is separated from the arterial lumen by a fibrous cap and myeloproliferative tissue that consists of extra-cellular matrix and smooth muscle cells. The junction between the cap and the morphologically more normal area of artery is known as the “shoulder” region of the atherosclerotic plaque. This area is typically more cellular than other areas of the plaque and may contain a variable composition of smooth muscle cells, macrophages, and even T-cells. The shoulder region is most prone to rupture and may even contain evidence of previously healed fissures.

Early concepts of atherosclerosis involved progressive luminal narrowing until the blood flow was compromised to the point that organ metabolic needs could no longer be met, producing ischemia and infarction of the subtended tissue such as the heart or the brain. Over the last 15 years, this concept has changed dramatically to include the notion of plaque rupture and fissuring in the artery as the cause of compromised blood flow precipitating clinical events such as myocardial infarction and crescendo angina. Therefore, clinical events are now thought to be the consequence of an abrupt, catastrophic change in plaque morphology rather than a gradual narrowing of the lumen. Evidence for plaque rupture can also be found in patients dying from non-cardiac causes, suggesting that plaque rupture is part of atherosclerotic lesion progression rather than a unique feature of clinical events from atherosclerosis.

Given that plaque rupture is implicated as a precipitating event in the clinical manifestations of atherosclerosis, a considerable effort has been directed at understanding the events involved in this process. Mature atherosclerotic plaques can be categorized as either stable or vulnerable to rupture. Stable plaques tend to be characterized by a smaller lipid core, a thick fibrous cap, and shoulder regions with few inflammatory cells, whereas vulnerable plaques contain considerable lipid in their core, a thin fibrous cap, and a robust population of macrophages and T-cells in their shoulder regions. These differences in morphology suggest that vulnerable plaques may be weaker structurally and more likely to rupture in response to the physical forces of flowing blood. This contention is supported by experimental data linking an increased content of macrophages in lesions to structural weakness.

In summary, atherosclerosis is characterized by LDL deposition in the arterial wall, a process that is stimulated by environmental and genetic factors such as tobacco use, diabetes and hypertension. This LDL deposition occurs primarily within macrophages and ultimately begets the formation of well-defined lesions in the arterial intima. The accumulation of macrophages reflects the inflammatory component of atherosclerosis. Such lesions then develop and macrophage-rich lesions are prone to rupture and, as a consequence, can precipitate the clinical events such as heart attack and stroke.

### Endothelial Function/Dysfunction

The precipitation of acute vascular events in atherosclerosis involves processes that go beyond plaque vulnerability and rupture. There is now a growing appreciation that local homeostatic processes in the arterial wall are also abnormal in those patients with frank atherosclerosis and risk factors for atherosclerosis. Among the more important components of vascular homeostasis is the endothelium, as it serves as the interface between the vascular wall and flowing blood. Through the release of autocrine and paracrine factors, the endothelium regulates a number of important processes such as vascular tone, platelet adhesion, and leukocyte transit into tissues and the vascular wall. The principal factors released by the endothelium that regulate vascular homeostasis on a moment-by-moment basis are prostacyclin, leukotrienes, and nitric oxide.

Endothelial production of nitric oxide is important in the regulation of vascular tone, arterial pressure, platelet adhesion, and leukocyte trafficking, as mice lacking endothelial nitric oxide synthase (the enzyme that generates nitric oxide in endothelial cells) exhibit spontaneous hypertension, defective vascular remodeling, enhanced vascular thrombosis and leukocyte interactions. The “classic” model of bioactivity of nitric involves its binding to the heme group of guanylate cyclase in target cells (e.g., platelets, smooth muscle cells) to increase cellular cGMP and activate cGMP-dependent protein kinase thereby effecting nitric oxide-mediated vasodilation and platelet inhibition.

The term endothelial dysfunction as used herein refers to a loss of normal homeostatic functions (e.g., vasodilatation, platelet inhibition). This condition often occurs early in the course of atherosclerosis with one important manifestation being a reduction in the bioactivity of endothelium-derived nitric oxide. Although the loss of nitric oxide bioactivity is not the only manifestation of endothelial dysfunction, it is an independent predictor of future cardiovascular events in patients with atherosclerosis. There are many potential reasons for impaired nitric oxide bioactivity. These range from inadequate nitric oxide production to nitric oxide degradation or an inadequate response to nitric oxide. There is evidence to support defects in all facets of nitric oxide production and metabolism in the setting of vascular disease, but oxidative events figure prominently in many studies of impaired nitric oxide bioactivity.

Attempts have been made to understand those factors that trigger a lipid deposit resulting in the eventual occlusion of a vessel (stenosis). According to the ‘oxidative modification theory’ of atherosclerosis, the oxidation of LDL predominantly occurs in the sub-endothelial space of the vessel wall. Oxidized LDL is pro-atherogenic by promoting the accumulation of lipids in cells, disturbing the normal vasoregulatory function of endothelial cells, being cytotoxic to endothelial and other cells, mediating the generation of a necrotic core, promoting the recruitment of inflammatory cells, and by inducing thrombogenic tissue factor and the expression of adhesion molecules on endothelial cells. Accumulation of lipid by macrophages can induce the secretion of matrix metalloproteinases and cytokines (e.g., interleukin-8). These thrombotic, adhesive and inflammatory properties of oxidized LDL may be critical for disease progression (whether episodic or continuous) and likely involves episodic damage to the endothelium.

According to the “response to injury” hypothesis of atherosclerosis, endothelial cell injury can itself trigger or contribute to the development of atherosclerosis.

### Restenosis

Re-endothelialization is the process whereby an intact endothelial cell layer grows back over a previously denuded area of the blood vessel. Commonly, the re-growth of endothelial cells is initiated at branching points of smaller vessels and cell growth then progresses into the larger vessel. Re-endothelialization is not identical to the process of endothelial cell proliferation. The former is limited to previously damaged areas, whereas endothelial cell proliferation is a more general process required, for instance in angiogenesis which itself can promote rather than inhibit atherosclerosis (Circulation 1999; 99:1726-1732).

Re-endothelialization is particularly important for the prevention of restenosis after BA (where the endothelial cell layer of large areas of vessels become removed). For example, the local delivery of vascular endothelial growth factor (a growth factor that specifically promotes the growth of endothelial cells) accelerates re-endothelialization and attenuates intimal hyperplasia in the balloon-injured rat carotid artery (Circulation 1995; 91:2793-2801). Re-endothelialization may also be important in atherosclerosis where injury to endothelial cells occurs, for example as a result of the accumulation and toxic properties of oxidized LDL.

The endothelium is a cell layer that lines internal body surfaces such as in the heart, blood and lymphatic vessels and other fluid filled cavities and glands. Endothelium must be induced to re-grow if the integrity of the surface is to be maintained. The integrity of endothelium in blood vessels is of central importance to vascular homeostasis in general and processes related to restenosis and atherosclerosis in particular. The latter include, but are not limited to, the control of vascular tone via endothelium-dependent relaxing factor (i.e., nitric oxide produced by endothelial nitric oxide synthase), the deposition of matrix by, and proliferation of, smooth muscle cells, the infiltration of the vessel wall by inflammatory blood cells, and the control of coagulation and platelet aggregation. Smooth muscle cell proliferation is often implicated in restenosis. Prevention of the proliferation has been effective in inhibiting the progress of restenosis. However, the direct general prevention of smooth muscle cell proliferation may not always be beneficial, as for instance it may decrease the stability of plaques and thereby promote clinical events by promoting plaque rupture.

Promotion of re-endothelialization may overcome gross endothelial dysfunction (due to loss of endothelial cells). Methods of promoting re-endothelialization may also extend to methods of treating conditions associated with endothelial dysfunction, for example, in the control of vascular tone via endothelium-dependent relaxing factor (i.e., nitric oxide produced by endothelial nitric oxide synthase), the deposition of matrix by, and proliferation of, smooth muscle cells, the infiltration of the vessel wall by inflammatory blood cells, and the control of coagulation and platelet aggregation.

### Formulations

In another aspect the present invention also relates to pharmaceutical compositions comprising at least one compound of Formula (I) as defined herein together with pharmaceutically acceptable excipients, adjuvants and/or diluents.

In a further aspect the invention relates to a process for preparing a pharmaceutical composition comprising homogeneously mixing a compound of Formula (I) with a pharmaceutically acceptable adjuvant, diluent and/or carrier.

In accordance with the present invention, when used for the treatment or prevention of cardiovascular diseases or disorders, compound(s) of Formula (I) may be administered alone. Alternatively, the compounds may be administered as a pharmaceutical or veterinary formulation comprising one or more compound(s) of Formula (I). The compound(s) may be present as suitable salts, including pharmaceutically acceptable salts.

Also in accordance with the present invention, the compounds of Formula (I) may be used in combination with other known agents and treatment regimes. For example, the compounds of Formula (I) may be used in combination with other agent(s) used for treating cardiovascular disease. These agent(s) may include lipid-lowering drugs such as statins (e.g., simvastatin, pravastatin, lovostatin, and the like), blood pressure-lowering drugs such as Angiotensin Converting Enzyme (ACE) inhibitors (e.g., perindopril, ramipril, etc), beta blockers, diuretics, calcium channel blockers, etc, and agents which promote induction of heme-oxygenase 1 (HO-1). General classes and examples of agents for treating cardiovascular include those disclosed in Martindale, the Extra Pharmacopoeia, (thirty-first Edition), Ed. James E. F. Reynolds 1996, which is incorporated herein by cross-reference.

Combinations of active agents, including compounds of the invention, may be synergistic.

By pharmaceutically acceptable salt it is meant those salts which, within the scope of sound medical judgement, are suitable for use in contact with the tissues of humans and lower animals without undue toxicity, irritation, allergic response and the like, and are commensurate with a reasonable benefit/risk ratio. Pharmaceutically acceptable salts are well known in the art.

For instance, suitable pharmaceutically acceptable salts of compounds according to the present invention may be prepared by mixing a pharmaceutically acceptable acid such as hydrochloric acid, sulfuric acid, methanesulfonic acid, succinic acid, fumaric acid, maleic acid, benzoic acid, phosphoric acid, acetic acid, oxalic acid, carbonic acid, tartaric acid, or citric acid with the compounds of the invention. Suitable pharmaceutically acceptable salts of the compounds of the present invention therefore include acid addition salts.

S. M. Berge et al. describe pharmaceutically acceptable salts in detail in J. Pharmaceutical Sciences, 1977, 66:1-19. The salts can be prepared in situ during the final isolation and purification of the compounds of the invention, or separately by reacting the free base function with a suitable organic acid. Representative acid addition salts include acetate, adipate, alginate, ascorbate, aspartate, benzenesulfonate, benzoate, bisulfate, borate, butyrate, camphorate, camphorsulfonate, citrate, digluconate, cyclopentanepropionate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium potassium, calcium, magnesium, and the like, as well as non-toxic ammonium, quaternary ammonium, and amine cations, including, but not limited to ammonium, tetramethylammonium, tetraethylammonium, methylamine, dimethylamine, trimethylamine, triethylamine, ethylamine, triethanolamine and the like.

Convenient modes of administration include injection (subcutaneous, intravenous, etc.), oral administration, inhalation, transdermal application, topical creams or gels or powders, or rectal administration. Depending on the route of administration, the formulation and/or compound may be coated with a material to protect the compound from the action of enzymes, acids and other natural conditions which may inactivate the therapeutic activity of the compound. The compound may also be administered parenterally or intraperitoneally.

Dispersions of compounds according to the invention may also be prepared in glycerol, liquid polyethylene glycols, and mixtures thereof and in oils. Under ordinary conditions of storage and use, pharmaceutical preparations may contain a preservative to prevent the growth of microorganisms.

Pharmaceutical compositions suitable for injection include sterile aqueous solutions (where water soluble) or dispersions and sterile powders for the extemporaneous preparation of sterile injectable solutions or dispersions. Ideally, the composition is stable under the conditions of manufacture and storage and may include a preservative to stabilise the composition against the contaminating action of microorganisms such as bacteria and fungi.

In one embodiment of the invention, the compound(s) of the invention may be administered orally, for example, with an inert diluent or an assimilable edible carrier. The compound(s) and other ingredients may also be enclosed in a hard or soft shell gelatin capsule, compressed into tablets, or incorporated directly into an individual's diet. For oral therapeutic administration, the compound(s) may be incorporated with excipients and used in the form of ingestible tablets, buccal tablets, troches, capsules, elixirs, suspensions, syrups, wafers, and the like. Suitably, such compositions and preparations may contain at least 1% by weight of active compound. The percentage of the compound(s) of formula (I) in pharmaceutical compositions and preparations may, of course, be varied and, for example, may conveniently range from about 2% to about 90%, about 5% to about 80%, about 10% to about 75%, about 15% to about 65%; about 20% to about 60%, about 25% to about 50%, about 30% to about 45%, or about 35% to about 45%, of the weight of the dosage unit. The amount of compound in therapeutically useful compositions is such that a suitable dosage will be obtained.

The language “pharmaceutically acceptable carrier” is intended to include solvents, dispersion media, coatings, anti-bacterial and anti-fungal agents, isotonic and absorption delaying agents, and the like. The use of such media and agents for pharmaceutically active substances is well known in the art. Except insofar as any conventional media or agent is incompatible with the compound, use thereof in the therapeutic compositions and methods of treatment and prophylaxis is contemplated. Supplementary active compounds may also be incorporated into the compositions according to the present invention. It is especially advantageous to formulate parenteral compositions in dosage unit form for ease of administration and uniformity of dosage. “Dosage unit form” as used herein refers to physically discrete units suited as unitary dosages for the individual to be treated; each unit containing a predetermined quantity of compound(s) is calculated to produce the desired therapeutic effect in association with the required pharmaceutical carrier. The compound(s) may be formulated for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

In one embodiment, the carrier may be an orally administrable carrier.

Another form of a pharmaceutical composition is a dosage form formulated as enterically coated granules, tablets or capsules suitable for oral administration.

Also included in the scope of this invention are delayed release formulations.

Compounds of the invention may also be administered in the form of a “prodrug”. A prodrug is an inactive form of a compound which is transformed in vivo to the active form. Suitable prodrugs include esters, phosphonate esters etc, of the active form of the compound.

In one embodiment, the compound may be administered by injection. In the case of injectable solutions, the carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by including various anti-bacterial and/or anti-fungal agents. Suitable agents are well known to those skilled in the art and include, for example, parabens, chlorobutanol, phenol, benzyl alcohol, ascorbic acid, thimerosal, and the like. In many cases, it may be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminium monostearate and gelatin.

Sterile injectable solutions can be prepared by incorporating the analogue in the required amount in an appropriate solvent with one or a combination of ingredients enumerated above, as required, followed by filtered sterilisation. Generally, dispersions are prepared by incorporating the analogue into a sterile vehicle which contains a basic dispersion medium and the required other ingredients from those enumerated above.

Tablets, troches, pills, capsules and the like can also contain the following: a binder such as gum gragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin or a flavouring agent such as peppermint, oil of wintergreen, or cherry flavouring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the analogue, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the analogue can be incorporated into sustained-release preparations and formulations.

Preferably, the pharmaceutical composition may further include a suitable buffer to minimise acid hydrolysis. Suitable buffer agent agents are well known to those skilled in the art and include, but are not limited to, phosphates, citrates, carbonates and mixtures thereof.

Single or multiple administrations of the pharmaceutical compositions according to the invention may be carried out. One skilled in the art would be able, by routine experimentation, to determine effective, non-toxic dosage levels of the compound and/or composition of the invention and an administration pattern which would be suitable for treating the diseases and/or infections to which the compounds and compositions are applicable.

Further, it will be apparent to one of ordinary skill in the art that the optimal course of treatment, such as the number of doses of the compound or composition of the invention given per day for a defined number of days, can be ascertained using convention course of treatment determination tests.

Generally, an effective dosage per 24 hours may be in the range of about 0.0001 mg to about 1000 mg per kg body weight; for example, about 0.001 mg to about 750 mg per kg body weight; about 0.01 mg to about 500 mg per kg body weight; about 0.1 mg to about 500 mg per kg body weight; about 0.1 mg to about 250 mg per kg body weight; or about 1.0 mg to about 250 mg per kg body weight. More suitably, an effective dosage per 24 hours may be in the range of about 1.0 mg to about 200 mg per kg body weight; about 1.0 mg to about 100 mg per kg body weight; about 1.0 mg to about 50 mg per kg body weight; about 1.0 mg to about 25 mg per kg body weight; about 5.0 mg to about 50 mg per kg body weight; about 5.0 mg to about 20 mg per kg body weight; or about 5.0 mg to about 15 mg per kg body weight.

Alternatively, an effective dosage may be up to about 500 mg/m². For example, generally, an effective dosage is expected to be in the range of about 25 to about 500 mg/m², about 25 to about 350 mg/m², about 25 to about 300 mg/m², about 25 to about 250 mg/m², about 50 to about 250 mg/m², and about 75 to about 150 mg/m².

In another embodiment, a compound of Formula (I) may be administered in an amount in the range from about 100 to about 1000 mg per day, for example, about 200 mg to about 750 mg per day, about 250 to about 500 mg-per day, about 250 to about 300 mg per day, or about 270 mg to about 280 mg per day.

The invention will now be described in more detail, by way of illustration only, with respect to the following examples. The examples are intended to serve to illustrate this invention and should not be construed as limiting the generality of the disclosure of the description throughout this specification.

### EXAMPLES

### Example 1

### Probucol Inhibits Atherosclerosis in Apolipoprotein E Gene Knock-Out Mice without Inhibition of Lipoprotein Oxidation in the Vessel Wall

This example illustrates that the anti-atherosclerotic activity of probucol is not due to inhibition of lipoprotein lipid oxidation in the vessel wall.

### Materials and Methods

Chemicals were obtained from Sigma (St. Louis, Mo.), except α-tocopherol was a gift from Henkel Corporation (Sydney, Australia), cholest-5-en-3β-ol-7-one (7-ketocholesterol, 7KC) was purchased from Steraloids Inc. (Wilton, N.H.), and probucol from Medichem (Barcelona, Spain). Hydroperoxide of cholesteryllinoleate (C18:2) was prepared as described in Methods in Enzymology 1994, 233:469-489, and used as standard for cholesterylester hydroperoxides (CE—OOH). Buffers were prepared from nanopure water, stored over Chelex-100® (BioRad, Richmond, Calif.) to remove contaminating transition metals, filtered and argon-flushed immediately prior to use.

ApoE−/− mice

Male C57BL/6J mice, homozygous for the disrupted apoE gene (apoE−/−) and originally purchased from Jackson Laboratories (Bar Harbor, Me.), were used at 8-10 weeks of age and then fed for 24 weeks ad libitum a high fat diet containing 21.2 (w/w) fat and 0.15% (w/w) cholesterol (specifications of the Harlan Teklad diet TD88137), without (controls, 103 mice) or with probucol (1% w/w, 87 mice), as described in Arteriosclerosis Thrombosis and Vascular Biology 2000, 20:e26-e33. The local animal ethics committee approved the study.

### Aortic Sampling for Biochemical and Histological Analyses

Procedures were carried out as described in Arteriosclerosis Thrombosis and Vascular Biology 2000, 20:e26-e33. For biochemistry, hearts and aortas past the femoral bifurcation were excised carefully (n=86 and 70 for control and probucol animals, respectively), cleaned, placed immediately in ice-cold buffer containing protease inhibitors and antibiotics, and then stored at −80° C. For histology, separate mice (n=17 each, for controls and probucol) were perfusion fixed, the hearts and aortas dissected and processed for blinded morphometry at the sinus, arch and descending thoracic and abdominal aorta, precisely as described in Arteriosclerosis Thrombosis and Vascular Biology 2000, 20:e26-e33. Mean areas from thoracic and abdominal aortas were similar and therefore combined and presented as a single value. For histology, sections at the sinus were taken ˜200 μm from the first appearance of the leaflets. Sections at the thoracic and abdominal aorta were taken at the branch point of the 3^{rd} pair of intra-costal arteries and the celiac artery, respectively.

### Aortic Biochemistry

Aortas and hearts isolated from control and probucol-treated animals were separated into two groups, one for F₂-isoprostanes and arachidonic acid determination (n=10 for each, control and probucol) and the other for total cholesterol, C, 7KC, CE, CE-OOH, α-tocopherol, and probucol (n=76 and 60 for control and probucol, respectively). On the day of analysis, samples were thawed and each divided into three segments: the aortic sinus (S), arch (A) and thoracic plus abdominal aorta (T+A=remaining aorta). For the sinus material the origin of the aorta was dissected from the surrounding myocardium and used for analysis, whereas the arch was defined as from where the aorta leaves the heart to just distal to the right subclavian artery. For F₂-isoprostanes and arachidonic acid determination, each aortic segment was analysed individually. For all other biochemical analytes, respective individual aortic segments were pooled (n=19 and 15 segments per control and probucol pool, respectively) and then analysed as separate pools (n=4 for control and probucol).

For F₂-isoprostanes, the thawed aortic segment (≈20 mg wet weight) was blotted dry, weighed and F₂-isoprostanes analyzed by electron capture negative ionization GC/MS after solid-phase extraction and HPLC purification as described in Analytical Biochemistry 1999, 268:117-125, using [D₄]-8-iso-prostaglandin F_{2α} (Cayman Chemical) as internal standard. For arachidonate, phospholipids were separated by thin-layer chromatography, the fatty acid methyl esters then prepared and analyzed by GLC, as described in American Journal of Clinical Nutrition 2000, 71:1085-1094.

For other analytes, pooled aortic segments were homogenized and extracted as described in Journal of Lipid Research 1999, 40:1104-1112, and the organic phase analysed by HPLC with electrochemical (for α-tocopherol), UV (C and CE) and post-column chemiluminescence detection (CE-OOH) as described in Methods in Enzymology 1999, 299:362-375. CE-OOH were measured as a marker of primary lipoprotein lipid peroxidation as they are the primary and major lipid oxidation products formed in lipoproteins from apoE−/− mice undergoing oxidation in the presence of α-tocopherol. For total cholesterol and 7KC, separate 10 μL and 100 μL aliquots, respectively, of the re-dissolved organic extracts were saponified, and subjected to HPLC as described in Journal of Lipid Research 1997, 38:1730-1745. For 7KC, a silica column (0.46×15 cm, 120 Å, 5 μm, Supelco) with guard column (3 μm particle size) was eluted with hexane/isopropylalcohol/acetonitrile (94.8:4.6:0.6 v/v/v) at 1.0 mL/min and monitored at 234 nm. For all chromatographic analyses, compounds were quantified by area comparison using authentic standards.

### Statistics

Data on lesion size are presented as mean±SEM and the effects of probucol analysed by the Mann-Whitney U-test. Biochemical parameters were compared using a one-way ANOVA or Mann-Whitney U-test. Data on total cell numbers, macrophages and collagen content are expressed as mean±SD, and significant differences between means evaluated using the Student's t-test. Statistical significance was accepted at p<0.05.

### Results

### Site-Specific Effect of Probucol on Atherosclerosis

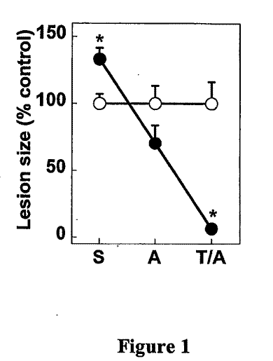

In apoE−/− mice, probucol affects atherosclerosis non-uniformly, as described previously in Arteriosclerosis and Thrombosis in Vascular Biology 2000, 20:e26-e33. The results of the present study, employing a large number of animals, confirmed this earlier observation. Probucol significantly increased lesion size by 33% at the sinus (0.68±0.35 and 0.90±0.56 mm² for control and probucol, respectively, p<0.01), while it visibly inhibited atherosclerosis in other parts of the aorta, including the carotid and femoral arteries (Table 1, FIG. 1). Probucol increasingly inhibited disease along the aortic tree, with 36% inhibition at the arch (0.19±0.02 and 0.12±0.03 mm² for control and probucol, respectively, not significant) and 94% inhibition at the descending aorta (117,000±19,200 and 7,300±2,400 μm² for control and probucol, respectively, p<0.0001) (Table 1, FIG. 1).

### Effect of Probucol on Non-Oxidized Lipids and Lipid-Soluble Antioxidants

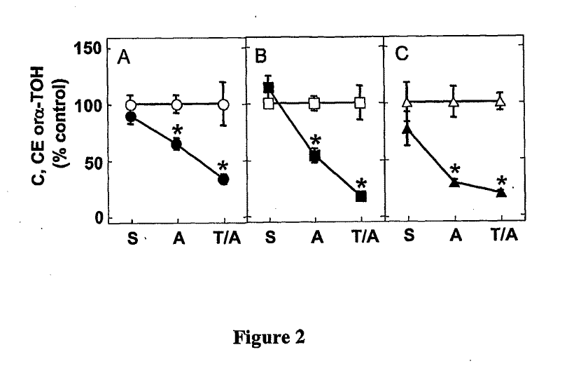

The ability of probucol to simultaneously promote and inhibit atherosclerosis provides an experimental model to directly relate the extent of lipoprotein lipid oxidation and atherogenesis in different aortic segments of the same animal. To do this, the concentrations of the non-oxidized lipids, C and cholesterylesters (CE, defined as the sum of C18:2 plus cholesterylarachidonate, C20:4), and the lipid-soluble antioxidant α-tocopherol as measures of lipoprotein lipid accumulation were determined. For control and probucol-treated animals, lesions at the sinus contained more C per protein than respective lesions at the arch and thoracic/abdominal aorta (Table 1). In contrast, the protein-standardized contents of C18:2, C20:4 and α-tocopherol were not different at the three sites in control animals, whereas probucol significantly decreased the tissue content of CE and the vitamin. FIG. 2 is a graphic representation of these results, with data from probucol-treated mice expressed relative to that of control animals for each of the three sites. As can be seen, compared to controls, probucol decreased the concentrations of C (FIG. 2A), CE (FIG. 2B) and α-tocopherol (FIG. 2C) in the arch and descending aorta, in parallel with inhibition of disease (FIG. 1). However, probucol did not increase the content of C (FIG. 2A), CE (FIG. 2B) and α-tocopherol (FIG. 2C) at the sinus.

TABLE 1
Total cell density, macrophage and extra-cellular matrix content in aortic sinus
lesions of probucol-treated and control apoE−/− mice

| | | Aortic Site | | |
|---|---|---|---|---|
| | | | | Thoracic/ |
| Parameter | Treatment | Sinus | Arch | Abdominal |
| Lesion size (μm² × 10⁻³) | Control | 679 ± 35 | 189 ± 24^{a} | 117 ± 19^{a} |
| | Probucol | 904 ± 56 | 121 ± 27^{a} | 7 ± 2^{a} |

Lipids (nmol/mgp)

| | | | | |
|---|---|---|---|---|
| C | Control | 1033 ± 169 | 689 ± 107 | 643 ± 245^{a} |
| | Probucol | 928 ± 144 | 447 ± 71^{a} | 215 ± 55^{a} |
| C18:2 | Control | 57 ± 3 | 65 ± 6 | 64 ± 22 |
| | Probucol | 61 ± 11 | 33 ± 9^{a} | 10 ± 5^{a,b} |
| C20:4 | Control | 26 ± 3 | 28 ± 6 | 26 ± 6 |
| | Probucol | 33 ± 6 | 17 ± 3^{a} | 5.0 ± 1.4^{a,b} |

Antioxidants (nmol/mgp)

| | | | | |
|---|---|---|---|---|
| α-Tocopherol | Control | 4.0 ± 1.4 | 5.6 ± 1.9 | 6.1 ± 0.9 |
| | Probucol | 3.0 ± 1.2 | 1.6 ± 0.3^{a} | 1.2 ± 0.2^{a} |

Oxidized lipids

| | | | | |
|---|---|---|---|---|
| CE-OOH | Control | 115.0 ± 37.8 | 409.2 ± 357.8 | 39.1 ± 11^{a} |
| (pmol/mgp) | Probucol | 88.1 ± 26.6 | 143.8 ± 118.7 | 20.3 ± 8.1 |
| CE-OOH/CE | Control | 1.38 ± 0.40 | 4.56 ± 3.83 | 0.45 ± 0.14^{a,b} |
| (mmol/mol) | Probucol | 0.93 ± 0.12 | 2.70 ± 2.04 | 1.46 ± 0.72 |
| 7KC | Control | 0.55 ± 0.28 | 0.52 ± 0.21 | 0.40 ± 0.17 |
| (pmol/mgp) | Probucol | 0.21 ± 0.11 | 0.20 ± 0.11 | 0.10 ± 0.04 |
| 7KC/total | Control | 0.30 ± 0.04 | 0.32 ± 0.14 | 0.26 ± 0.10 |
| cholesterol | Probucol | 0.16 ± 0.06 | 0.25 ± 0.20 | 0.08 ± 0.02 |
| (mmol/mol) | | | | |
| F₂-isoprostanes | Control | 0.75 ± 0.40 | 0.42 ± 0.11 | 0.16 ± 0.03^{a} |
| (pmol/mgp) | Probucol | 0.39 ± 0.11 | 0.35 ± 0.11 | 0.14 ± 0.04^{a,b} |
| F₂-isoprostanes | Control | 14.4 ± 6.5 | 8.4 ± 4.2^{a} | 2.4 ± 0.4^{a,b} |
| (μmol/mol | Probucol | 8.6 ± 4.8 | 10.4 ± 3.6 | 3.8 ± 1.4^{a,b} |
| arachidonate) | | | | |

Lesion data show mean ± SEM from 17 mice per group.
Biochemical data show mean ± SD from four separate pools each containing 19 (control) and 15 (probucol) respective sections, except for F₂-isoprostanes that show mean ± SD of ten individual sections.
CE represents C18:2 plus C20:4.
^{a,b}Significantly different from sinus and arch, respectively.

### Effect of Probucol on Lipid Oxidation in Atherosclerotic Lesions at Different Sites

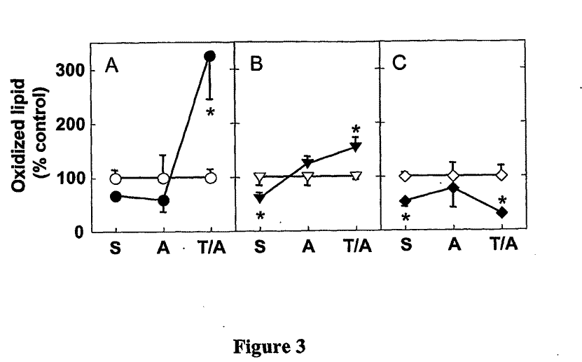

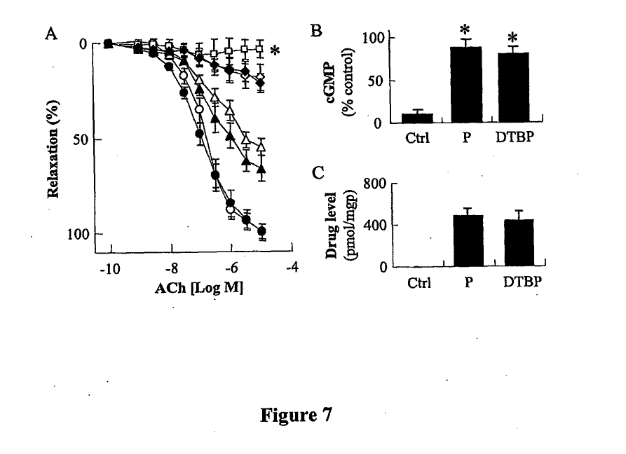

Three separate measures were used to assess lipid oxidation, i.e., CE-OOH, F₂-isoprostanes and 7KC. Of these, CE-OOH were more abundant than 7KC and F₂-isoprostanes (Table 1). In control and probucol-treated mice, tissue 7KC was not different at different sites, irrespective of whether data was standardized to protein or parent lipid. In contrast, protein- and parent lipid-standardized concentrations of CE-OOH and F₂-isoprostanes were decreased at the thoracic/abdominal site compared with aortic sinus (Table 1). FIG. 3 compares the parent lipid-standardized content of oxidized lipids at the three sites in control versus probucol-treated mice. At the sinus, where probucol increased lesion size (FIG. 1), the drug decreased the concentrations of CE-OOH (FIG. 3A), F₂-isoprostanes (FIG. 3B) and 7KC (FIG. 3C), and this reached statistical significance in the case of F₂-isoprostanes and 7KC. In contrast, probucol significantly increased CE-OOH and F₂-isoprostanes at the descending aorta where the drug almost completely prevented atherosclerosis. All three parameters of lipid oxidation are expressed relative to the respective parent molecule (i.e., CE for CE-OOH, arachidonate for F₂-isoprostanes, and total cholesterol for 7KC) to distinguish lipid oxidation from lipid load, as the latter was affected significantly by probucol (FIG. 2A, B). However, even when the lipid oxidation parameters were expressed per protein, their concentrations did not reflect the effect of probucol on lesion development (not shown).

### Example 2

### Probucol Inhibits Atherosclerosis in Apolipoprotein E−/− Mice Via an Anti-Inflammatory Activity

This example illustrates that the anti-atherosclerotic activity of probucol is due to its anti-inflammatory activity, and that probucol alters the composition of atherosclerotic lesions such that they change from a rupture prone, pro-inflammatory to a more stable, fibrotic type.

### Materials and Methods

The materials and methods used were essentially as described under Example 1. In addition, for total cell numbers, nuclei were counted in hematoxylin and eosin-stained sections and expressed per lesion area. At the sinus, sections were taken ˜200 μm from the first appearance of the leaflets. Sections at the thoracic and abdominal aorta were taken at the branch point of the 3^{rd} pair of intra-costal arteries and the celiac artery, respectively. For macrophages, 4 μm paraffin sections were deparaffinized, rehydrated and endogenous peroxidase quenched with 3% hydrogen peroxide (15 min). Enzymatic antigen retrieval was performed in trypsin (1 mg/mL) solutions pH 7.7 containing 4 mM CaCl₂ and 200 mM Tris for 30 min at 37° C., followed by a 20 min incubation in 5% normal rabbit blocking serum. Sections were then incubated overnight in a humidified chamber and at 4° C. with monoclonal rat-anti-mouse F4/80 antibody (Caltag Laboratories; dilution 1:20), followed by biotinylated rabbit anti-rat IgG (Vector Laboratories; dilution 1:200, 30 min), Vectorstain Elite ABC reagent (Vectorstain Elite ABC Kit, Vector Laboratories; 30 min), and 3,3′-diaminobenzidine substrate-chromogen (Dako Corporation) with counterstaining using Harris hematoxylin. Images were captured using a Zeiss Axiophot Photomicroscope, and the area staining positive for F4/80 antigen expressed as a percentage of the total lesion area. Collagen was determined as described in Histochemical Journal 1979, 11:447-455. Briefly, 4 μm sections were deparaffinized, rehydrated and stained with 0.1% Sirius red (Fast red F3B) in saturated aqueous picric acid (pH 2.0) for 1 h at room temperature and then transferred to a solution of 0.01 N HCl for 2 min followed by counterstaining in Harris hematoxylin for 1 min. Total lesion area were measured from bright field images captured with an Olympus BX60 photomicroscope attached with a SPOT digital camera, whereas the birefringent area staining positive for Sirius red was detected using polarization microscopy and expressed as a percentage of the total lesion area at that site.

### Results

### Histological Assessment of Aortic Sinus Lesions

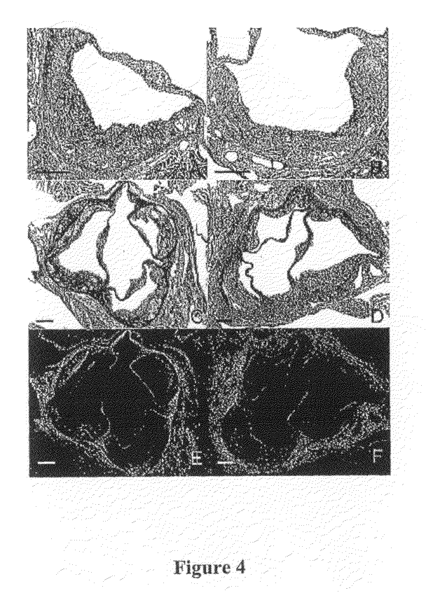

The results presented in Example 1 show that neither differences in lipid accumulation nor the extent of lipid oxidation could explain why lesions at the aortic sinus from probucol-treated mice were larger than those from control animals. Therefore, the cellular composition at different sites was determined. At the sinus, total cell numbers were similar in control and probucol-treated animals, so that probucol significantly decreased the number of cells per lesion area (Table 2). Similarly, probucol significantly decreased the percentage of lesion area covered by macrophages by nearly 50% (Table 2) (FIGS. 4A&B). In contrast, probucol significantly increased the percentage lesion area that stained positive for collagen (FIG. 4C-F). Thus, extra-cellular matrix accounted for 66±13 and 45±10% of the lesion area in the sinus of probucol-treated and control mice, respectively. At the descending aorta, probucol decreased total cell numbers and macrophages by ˜91%, a value comparable to the extent of lesion inhibition (Table 2). This dramatic change did not translate into a significant decrease in cells per lesion area, as lesions in the descending aorta of apoE−/− mice are less developed than those at the sinus, and consisted almost entirely of macrophages (Table 2). Extracellular deposits of collagen were barely detectable, and probucol did not alter its content (Table 2).

TABLE 2
Total cell density, macrophage and extra-cellular matrix content in aortic sinus
lesions of probucol-treated and control apoE−/− mice

| | Sinus | | T/A | |
|---|---|---|---|---|
| | Control | Probucol | Control | Probucol |
| Total cell density | (n = 8) | (n = 8) | (n = 6) | (n = 6) |
| Lesion area (μm² × 10⁻³) | 611 ± 155 | 889 ± 254^{a} | 268 ± 180 | 22 ± 15^{a} |
| Total number of cells | 1528 ± 274 | 1423 ± 441 | 114 ± 80 | 10 ± 13^{a} |
| Total cells/μm² | 2.6 ± 0.8 | 1.7 ± 0.6^{a} | 0.41 ± 0.32 | 0.36 ± 0.39 |
| | Control | Probucol | Control | Probucol |
| Macrophages (Mφ) | (n = 6) | (n = 6) | (n = 6) | (n = 6) |
| Lesion area (μm² × 10⁻³) | 616 ± 90 | 886 ± 89^{a} | 268 ± 180 | 22 ± 15^{a} |
| Mφ area (μm² × 10⁻³) | 158 ± 31 | 127 ± 53 | 254 ± 190 | 21 ± 12^{a} |
| % Mφ of total lesion area | 26 ± 5 | 14 ± 5^{a} | 93.4 ± 7.3 | 99.4 ± 1.4 |
| | Control | Probucol | Control | Probucol |
| Extra-cellular matrix (ECM) | (n = 6) | (n = 6) | (n = 6) | (n = 6) |
| Lesion area (μm² × 10⁻³) | 555 ± 188 | 878 ± 153^{a} | 195 ± 74 | 13 ± 3^{a} |
| ECM area (μm² × 10⁻³) | 248 ± 101 | 577 ± 126^{a} | 3.6 ± 1.9 | 0.0 ± 0.0^{a} |
| % ECM of total lesion area | 45 ± 10 | 66 ± 13^{a} | 3.3 ± 1.8 | 0.0 ± 0.0^{a} |

Total cell numbers and the percentages of lesion comprised of macrophages (Mφ) and extra-cellular matrix (ECM) were determined as described in the Methods section. Data shown represent mean ± SD for the number of animals indicated.
^{a}Significantly different from corresponding control.

### Example 3

### The Anti-Atherosclerotic Activity of Probucol Relates to the Extent to which the Drug is Metabolized

This example illustrates that the extent to which probucol is metabolised to probucol bisphenol and its oxidized form, probucol diphenoquinone, relates to the extent to which the drug inhibits atherosclerosis in apoE−/− mice, consistent with the notion that probucol is a pro-drug.

### Materials and Methods

The materials and methods are as described under Example 1. In addition, 3,3′,5,5′-tetra-tert-butyl-4,4′-bisphenol (bisphenol, BP) was purchased from Polysciences (Warrington, Pa.) and 3,3′,5,5′-Tetra-tert-butyl-4,4′-diphenoquinone (diphenoquinone, DPQ) was prepared from the bisphenol as described in Tetrahedron Letters 1988, 29:677-680. The quantity of probucol, BP, and DPQ in aortic homogenates was determined by gradient reverse-phase high-pressure liquid chromatography (HPLC) as described in FASEB Journal 1999, 13:667-675.

### Results

### Tissue Levels of Probucol and Probucol Metabolites

Previous studies by Barnhart J W, Wagner E R and Jackson R L published in Antilipidemic drugs (Witiak D T, Newman H A I, Feller D R, eds.) Amsterdam: Elsevier; 1991, pp. 277-298, suggest that probucol is metabolised in vivo to BP and its oxidized form, DPQ. It was therefore assessed whether the site-specific effect of probucol on atherosclerosis in apoE−/− mice (FIG. 1) was related to the concentration of the drug and/or its metabolites in the vessel wall. The results in Table 3 show that both probucol and the total amount of the drug were significantly lower in the descending aorta than the sinus.

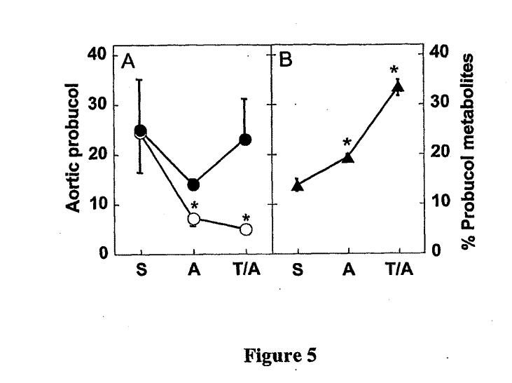

The lower concentration of probucol in the arch and thoracic/abdominal aorta compare to the sinus is not surprising, given that probucol is transported within lipoproteins, so that the results reflected the extent of lipoprotein infiltration at these sites. Consistent with this, the amount of probucol was no longer different for the different sites, when the drug concentration was standardized to C+CE (FIG. 5A), C or CE (data not shown) rather than protein (Table 3). In contrast, the concentration of probucol metabolites, i.e., BP plus DPQ, appeared to vary less than probucol itself at the three aortic sites, whether expressed per protein (Table 3) or lipid-adjusted (not shown).

TABLE 3
Aortic concentrations of probucol and its metabolites
BP and DPQ in apoE−/− mice after 24 weeks of intervention.

| | | Aortic Site | | |
|---|---|---|---|---|
| | | | | Thoracic/ |
| Parameter | Treatment | Sinus | Arch | Abdominal |
| Probucol | Probucol | 24.4 ± 8.1 | 7.1 ± 1.6^{a} | 4.9 ± 1.0^{a} |
| Probucol | Probucol | 3.9 ± 1.3 | 1.7 ± 0.4^{a} | 2.5 ± 1.0 |
| metabolites | | | | |
| Total Drug | Probucol | 28.3 ± 9.4 | 8.9 ± 2.0^{a} | 7.5 ± 1.9^{a} |

The data show mean ± SD from four separate pools each containing 19 (control) and 15 (probucol) respective sections.
^{a}Significantly different from sinus.

Importantly, when expressed relative to parent drug, the metabolites were significantly increased, and accounted for nearly one third of the drug, at the descending aorta (FIG. 5B), where atherosclerosis was inhibited compared with the aortic sinus where disease was enhanced (FIG. 1). Together, the results show that increased metabolism of probucol was associated with protection against atherosclerosis in apoE−/− mice.

### Example 4

### Identification of a Novel Pathway of Probucol Oxidation to a Biologically Active Intermediate

This example illustrates that probucol can be metabolised via a previously unrecognised pathway that yields bioactive intermediate(s) such as 4,4′-dithiobis(2,6-di-tert-butyl-phenol) (DTBP) that may contribute to vascular protection and anti-atherogenic activity.

### Materials and Methods

Materials: Probucol was obtained from Jucker Pharma (Stockholm, Sweden), and 3,3′,5,5′-tetra-tert-butyl-4,4′-bisphenol (BP), 4,4′-dithiobis(2,6-di-tert-butyl-phenol) (DTBP) and (2,2′-azobis(2-amidino-propane)-hydro chloride (AAPH) from Polysciences (Warrington, Pa.). Authentic DPQ was prepared from BP as described under Example 3, and purified by gradient reversed-phase HPLC (see below). Acetylcholine, lead dioxide (PbO₂) and ceric ammonium nitrate (purity 98%, a source of Ce⁴⁺) were obtained from Sigma ‘Dulbecco's’ phosphate buffered saline (DPBS, Sigma) was prepared from nanopure water and stored over Chelex-100® (BioRad, Richmond, Calif.) at 4° C. for 24 h to remove contaminating transition metals. All other reagents were of the highest quality available. Buffers were routinely filtered, argon-flushed and stored at 4° C. prior to use. Solutions of HOCl were prepared freshly before use by diluting reagent HOCl (Aldrich) into phosphate buffer (250 mM, pH 7.0) and standardizing with ε₂₃₅ nm˜100 M⁻¹cm⁻¹ (hypochlorous acid) and ε_{290 nm}˜300 M⁻¹cm⁻¹ (hypochlorite), as described in Journal of Physical Chemistry 1966, 70:3798-3805.
Animals: New Zealand White rabbits (2.5-3 kg) were obtained from a commercial farm (Wauchope, NSW Australia) and housed individually for the entire study period. Rabbits received normal chow (control) or chow supplemented with probucol (1%, w/w), DTBP (0.2%) or BP (0.02%); these concentrations resulted in comparable drug levels in the aortas of supplemented animals. Feed and water were provided ad libitum for a period of 4 weeks judged to be sufficient time for circulating drug levels to reach a maximum (data not shown). Animals were weighed weekly; mean body weights did not differ between treatment groups. The local ethics committee approved the study.
Vascular reactivity: Perfused rabbit aortas were harvested and vascular reactivity studies performed as described in Circulation 2003, 107:2031-2036. Briefly, within 2 h of isolation, ring segments ˜5 mm in length were mounted in a myobath system (World Precision Instruments, Sarasota, Fla.) containing 20 mL of Krebs solution aerated at 37° C. with 5% CO_{2(g)}, and the dilatory response of half maximally norepinephrine pre-constricted rings to incremental doses of ACh (10⁻⁹-10⁻⁵ mol/L) determined. Where indicated, reagent HOCl (final concentration 400 μM) was added to the Krebs solution and the rings incubated for 5 min prior to thorough washing, pre-constriction and assessment of vessel relaxation. In some studies rings were incubated with probucol or DTBP for 10 min, washed thoroughly and then exposed to HOCl prior to assessing endothelium-dependent relaxation. A maximum of three consecutive sequences of constriction/relaxation were performed for each ring.
Preparation of Aortic Homogenates: Aortic Rings Used in the Vascular Function Studies were removed and immediately cut into small pieces, frozen in liquid nitrogen, and then pulverized and homogenized as described. An aliquot (50 μL) of homogenate was removed for protein determination (BCA assay, Sigma) and the remainder extracted into methanol/hexane (5:1, v/v), the resulting hexane fraction dried and the residue suspended in isopropanol for analysis of probucol and its oxidation products. For cGMP determinations, aortic segments were treated with vehicle (control) or HOCl (400 μM) before addition of 1 μM ACh in the presence of 200 μM 3-isobutyl-1-methylxanthine and measurement of cGMP in the homogenate using a kit (Cayman Chemical, Ann Arbor, Mich.).
Probucol oxidation studies: Probucol is a highly lipophilic compound (partition coefficient between octanol and water=10 versus 10.8 for octanol) that readily distributes into lipoproteins. To mimic the biological situation, oxidation reactions were therefore performed with probucol (or DTBP) dissolved in hexane (2 mL) to which aliquots of HOCl were added to give the final concentrations indicated. The heterogeneous mixtures were shaken vigorously at 37° C. for 60 min, then placed on ice after adding 1 mL water, the hexane phase removed, dried under vacuum and resuspended in isopropanol (200 μL) for HPLC analyses (see below). The aqueous phase was analyzed for the content of sulfate anion (SO₄²⁻) by ion exchange chromatography (see below).
Analytical analyses: Probucol, DTBP, BP and DPQ were analyzed by gradient reversed-phase HPLC and quantified by peak area comparison with authentic standards. Under the conditions used, BP, DTBP, probucol and DPQ eluted at 12.5, 19.2, 20.8 and 28.4 min, respectively. Other oxidation products resolved by this HPLC system were also quantified by peak area comparison, using analytically pure samples obtained from semi-preparative (LC-18, 20 mm×25 cm, 5 μm) fractionation of the reaction mixture. Isolated samples were dried under vacuum, re-dissolved to a known concentration and assessed for purity by analytical HPLC (LC-18, 4.6 mm×25 cm, 5 μm) in combination with mass spectrometry (see below). The accumulation of DPQ was monitored at 420 nm, while all other products were monitored at 270 nm. Where required, ¹H-NMR (Bruker 600 MHz NMR spectrometer fitted with a standard hydrogen probe) was performed using authentic samples of DTBP and analytically pure DTBP isolated by HPLC after oxidation of probucol with HOCl.
Ion exchange chromatography was performed on a Waters IC PAK-A column (4.6×50 mm×10 μm) with an eluent containing sodium gluconate (0.32 g/L), boric acid (0.36 g/L), sodium tetraborate decahydrate (0.5 g/L), glycerol (5.0 mL/L), n-butanol (20 mL/L) and acetonitrile (120 mL/L) at a flow rate of 1.0 mL/min. Eluting anions were detected with a refractive index detector (limit of detection 10 μM), with sulfate anion eluting at ˜18 min identified by comparison with an authentic standard.
Mass spectrometry: Product masses were determined using electrospray ionization mass spectrometry (ESI-MS). Spectra were acquired using an API QStar Pulsar i hybrid tandem mass spectrometer (Applied Biosystems, Foster City Calif.). Samples (˜10 pmol, 1 μL) were dissolved in water/acetonitrile (1:4, v/v), loaded into nanospray needles (Proxeon, Denmark) and the tip positioned ˜10 mm from the orifice. Nitrogen was used as curtain gas and a potential of −800 V applied to the needle. A Tof MS scan was acquired (m/z 50-2000, 1 s) and accumulated for 1 min into a single file. Precursor masses determined from Tof MS scans were selected by Q1 for MS-MS analysis. Nitrogen was used as collision gas and a collision energy chosen that reduced the intensity of the precursor ion by ˜95%. Tandem mass spectra were accumulated into a single file for ˜2 min (m/z 50-1250).
Kinetic measurements: Kinetic determinations for the reactions of probucol and DTBP with HOCl were performed with an Applied Photophysics SX-17 MV stopped-flow spectrophotometer. Typically, 250 time-dependent spectra (logarithmic time-base, integration 2.56 ms, dead-time ˜2 ms and λ=350-750 nm, resolution 1 nm) were collected over 100 s at 25° C. Kinetic data were processed using Pro-Kineticist global analysis software (Pro-Kineticist, Version 4.1; Applied Photophysics: Leatherhead, U.K., 1996) as described in Chemical Research in Toxicology 2001, 14:1453-1464. Apparent rate constants (k) were then determined by linear regression. For these experiments probucol and DTBP were dissolved in 70% aqueous ethanol to enhance mixing with HOCl, as no meaningful kinetic data were obtained using the hexane/aqueous HOCl conditions described above.
Electronic spectroscopy: Electronic spectra were measured with a Hitachi UV/V is spectrophotometer. Spectra of authentic compounds and analytically pure oxidation products were obtained in ethanol (purity 99.7%) and maxima determined by manual peak picking.
Statistical analyses: Statistical analyses were performed using the Prism statistical program (GraphPad, San Diego, Calif.). Concentration-response curves were compared by two-way ANOVA. Student t-tests were performed to determine significant changes between paired data sets with Welch's correction employed for unequal variances where appropriate. In all cases, statistical significance was accepted at the 95% confidence interval (P<0.05).

### Results

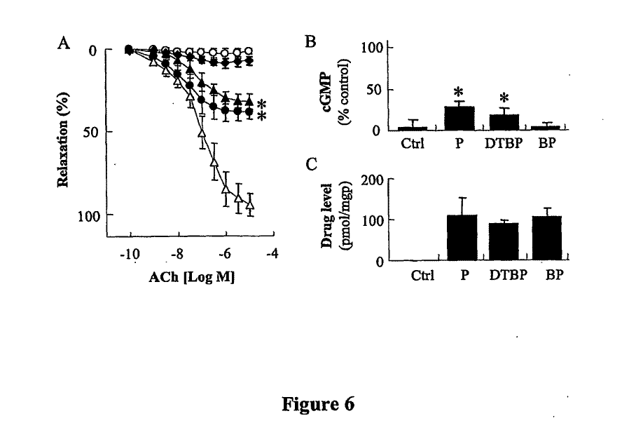

It has been reported that pre-treatment of aortic rings with non-cytotoxic concentrations of reagent HOCl (0-500 μM) resulted in a dose-dependent loss of endothelium-dependent relaxation (Arteriosclerosis Thrombosis and Vascular Biology 2004, 24, 2028-2033). Consistent with this, treating aortic rings from control rabbits with 400 μM HOCl essentially abolished subsequent relaxation in response to ACh (FIG. 6A). In contrast, rings from probucol-supplemented animals treated with HOCl retained responsiveness to ACh, as judged by their relaxation (FIG. 6A) and greater content of cGMP (FIG. 6B), although the extent of relaxation did not reach that of the native vessel without oxidant treatment (FIG. 6A). Aortas from probucol-fed rabbits contained probucol at ˜100 pmol/mg protein (FIG. 6C), demonstrating the presence of the drug in this tissue. Similar to the situation with in vivo supplemented probucol, pre-incubation of aortic rings from control animals with increasing amounts of added probucol for 10 min followed by thorough washing also protected the vessels from HOCl-induced loss of response to ACh in a concentration dependent manner, with full protection seen with 100 μM of the drug (FIG. 7). Rings pre-treated with 100 μM probucol responded to ACh by increased tissue content of cGMP (FIG. 7B), and contained ˜400 pmol drug/mg protein (FIG. 7C).

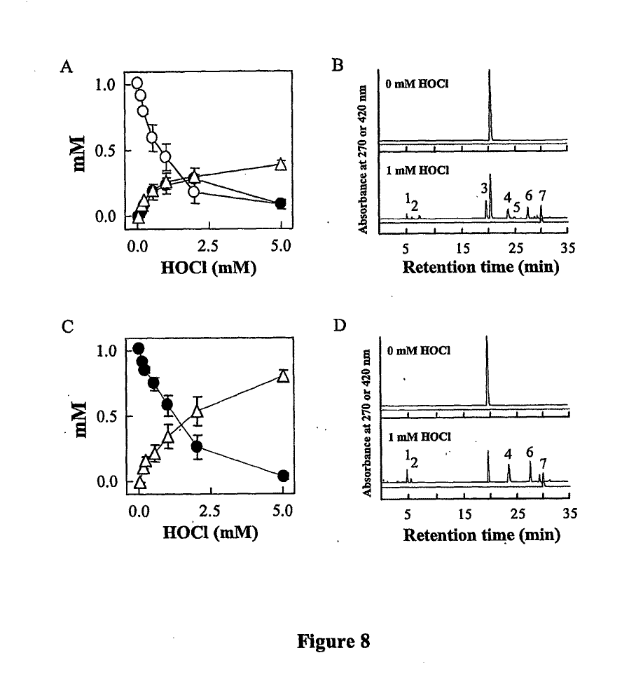

As a phenol, probucol is known to scavenge 1-electron (1e), i.e., radical oxidants, while little is known about its ability to scavenge 2-electron (2e) oxidants such as HOCl. Therefore, the oxidation of probucol by HOCl was examined. Reaction with HOCl resulted in the dose-dependent consumption of probucol as judged by HPLC (FIG. 8A).

TABLE 4
Negative ion mass analyses of isolated oxidation products
from in vitro reactions of probucol and HOCl^{a}

| Peak | | Expected | Observed |
|---|---|---|---|
| (retention | | m/z | m/z |
| time, min) | Product | (amu) | (amu) |
| 1 Chlorophenol (4.5) | 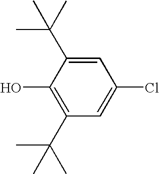 | 240 | 239 |
| 2 Sulfonic acid (5.5) | 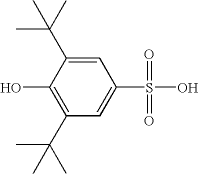 | 286 | 285 |
| 3 DTBP (19.4) | 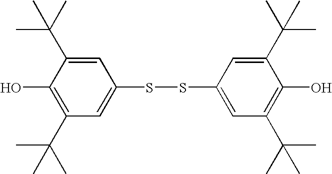 | 474 | 473 |
| 4 Thiosulfonate (22.9) | 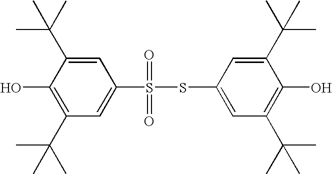 | 506 | 505 |
| 5 Disulfoxide (24) | 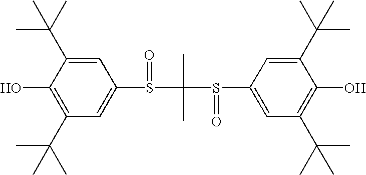 | 548 | 547 |
| 6 Disulfone (26.3) | 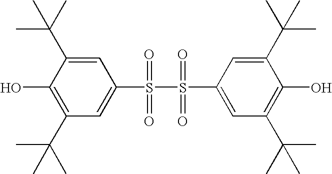 | 538 | 537 |
| 7 DPQ (29.2) | 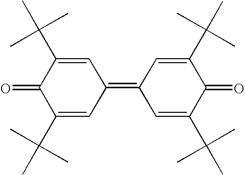 | 408 | Not assessed |

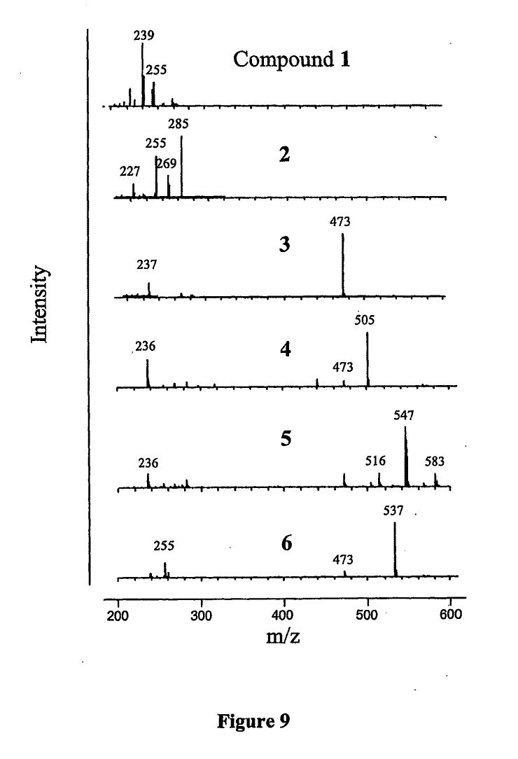

^{a}Analytically pure (>98% by HPLC) products were analyzed by negative ion ESI/MS that yields [M − H]− ions. The structure of 1 was confirmed by high resolution MS, showing the expected 3:1 chlorine isotope distribution (see FIG. 9)

This consumption occurred within minutes (data not shown), and resulted in the appearance of several oxidation products (compounds labelled 1-7 in FIG. 5B). Barnhart et al. (Journal of Lipid Research 1989, 30:1703-1710) reported oxidation of probucol to DPQ that co-eluted with 7 and appeared as a negative peak at 270 nm (FIG. 8B, solid line) and as a positive peak at 420 nm (FIG. 8B, dashed line), as described in Journal of Clinical Investigations 1999, 104:213-220.

Based on this, 7 was assigned as DPQ. Similarly, structural assignment for 3 was verified by spiking with authentic DTBP (not shown), mass (Table 3) and ¹H-NMR analyses of an analytically pure sample that showed singlet absorptions at 5.27, 7.33 and to 1.40 ppm in the ratio 1:2:18, assigned as phenolic, aromatic and methyl H-atoms, respectively. The identities of the remaining oxidation products were assigned by isolating sufficient analytically pure material for use in negative ion ESI-MS (FIG. 9). Table 4 summarizes the mass determinations of the isolated products 1-6. DTBP, DPQ and all other oxidation products were detected in samples of probucol oxidized with HOCl, independent of the mol ratio of oxidant to target (FIG. 8B). Structures were assigned to the various products based on experimentally determined molecular weights (Table 4) and known chemistry of sulfur-containing molecules. Oxidation product 1, assigned as a chlorophenol, showed the expected isotopic distribution for chlorine in the corresponding parent ion detected by mass spectrometry (FIG. 9). Using these assignments, the concentration of DTBP and ‘combined products’ (defined as DPQ plus 2, 4 and 6) were quantified retrospectively using corresponding authentic standards prepared from the isolated products. With HOCl at ≦2-mol oxidant per probucol, consumption of probucol was matched by a near stoichiometric accumulation of DTBP (filled circles) plus the combined products (open triangles) (FIG. 8A). Thereafter, the combined products increased slightly while DTBP decreased and was almost depleted with 5-mol HOCl per mol probucol.

As oxidation of probucol with HOCl consistently generated DTBP as a major intermediate over the oxidant concentrations tested (not shown), HOCl-induced oxidation of DTBP was examined. HOCl dose-dependently oxidized DTBP resulting in a near stoichiometric accumulation of the combined products (FIG. 8C) with a pattern similar to that for probucol (FIG. 8D). Notably, the aqueous phase of reaction mixtures containing probucol or DTBP oxidized with 5-mol equivalent HOCl also contained SO₄²⁻ at final concentrations of 139±14 or 204±43 nmol, respectively. By comparison, the concentration of SO₄²⁻ in the antioxidant-free mixtures containing hexane, water and HOCl was significantly lower (58±5 nmol) indicating that HOCl-mediated oxidation of probucol and DTBP produced SO₄²⁻.

To determine whether the probucol oxidation profile identified was specific to reactions with HOCl, additional 2e- and 1e-oxidants were tested (Table 5). As can be seen, in addition to HOCl, substantial oxidation (>90% consumption over 60 min) was observed with PbO₂, also a 2e-oxidant. By comparison, other 2e-oxidants, peroxynitrite (ONOO⁻) and hydrogen peroxide (H₂O₂), and the 1e-oxidants, Cu²⁺, Fe²⁺/H₂O₂ and the peroxyl radical generator AAPH caused little, and Ce⁴⁺ intermediate probucol consumption.

TABLE 5
Depletion of probucol or DTBP and corresponding yields of
DPQ by different 1e- and 2e-oxidants

| | Probucol | | DTBP | |
|---|---|---|---|---|
| | Depletion | DPQ Yield | Depletion | DPQ Yield |
| Oxidant | (%) | (μM) | (%) | (μM) |
| HOCl | 98 ± 4 | 102 ± 11 | 96 ± 3 | 91 ± 8.7 |
| ONOO⁻ | 7 ± 1 | 2.1 ± 0.2 | 6 ± 1 | 1.9 ± 0.6 |
| H₂O₂ | 0 ± 0 | 0 ± 0 | 0 ± 0 | 0 ± 0 |
| PbO₂ | 99 ± 1.1 | 92 ± 21 | 99 ± 0.4 | 145 ± 8.9 |
| Fe²⁺/H₂O₂ | 3 ± 1 | 8.7 ± 1.7 | 1 ± 0 | 4.8 ± 3.7 |
| AAPH | 0 ± 0 | 0 ± 0 | 0 ± 0 | 0 ± 0 |
| Cu²⁺ | 2 ± 1 | 0.9 ± 0.1 | 0 ± 0 | 0 ± 0 |
| Ce⁴⁺ | 15 ± 2 | 78 ± 7.9 | 16 ± 1 | 89 ± 11 |

Probucol or DTBP dispersed in 2 mL hexane (final concentration 1 mM) was treated with the oxidant indicated (final concentration 5 mM) for 60 min under air and at 37° C., the reaction mixture diluted with water (1 mL), extracted and the hexane phase analyzed as described in the Methods Section.
Peroxynitrite (ONOO⁻) was prepared, purified and standardized (ε₃₀₂ₙₘ ~1670 M⁻¹ · cm⁻¹) as described in Journal of Biological Chemistry 1991, 266: 4244-4250.
Abbreviations:
H₂O₂, hydrogen peroxide;
AAPH, 2,2′-azobis(2-amidino-propane)-hydrochloride, and
Ce⁴⁺, cerium (IV).

Two-electron oxidants generally gave higher yields of DPQ than 1e-oxidants, except for Ce⁴⁺. DTBP was detected consistently and to an extent proportional to the yield of DPQ, independent of whether 2e- or 1e oxidants were used (not shown). Similar to probucol, different oxidants converted DTBP to DPQ (Table 5), indicating DTBP was a likely intermediate in the oxidative conversion of probucol to DPQ initiated by 2e- and 1e-oxidants.

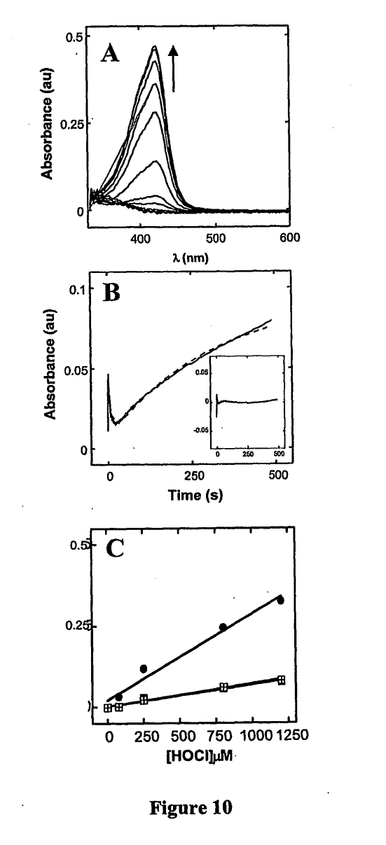

Oxidation of probucol and DTBP by HOCl was then analyzed by rapid scan absorbance spectrometry (FIG. 10). Oxidation resulted in the time-dependent increase in absorption at 440 nm, reflecting accumulation of DPQ (λₘₐₓ=440 nm) (FIG. 10 A). This optical change was used to determine the observed rate constants (k_{obs}) (FIG. 10B). Kinetic analyses indicated that probucol was oxidized in a biphasic process with the best fit to the data obtained using a simplified 3 species approach (a→b→c) to yield a low residual (FIG. 10B and inset). In contrast to probucol, DTBP was oxidized directly to DPQ (a→b) (data not shown). Plots of the observed rate constants versus HOCl concentration gave corresponding rate constants (FIG. 10C). Thus, probucol oxidized with rate constants k₁ and k₂ of 2.7±0.3×10² and 0.7±0.2×10² M⁻¹ s⁻¹, respectively, corresponding to the rapid and slow phase of DPQ accumulation. In contrast, DTBP was oxidized in a single rate-determining process with k=0.7±0.1×10² M⁻¹ s⁻¹.

As in vivo and in vitro added probucol protected aortic rings from HOCl-induced endothelial dysfunction, and HOCl converted probucol to DTBP that itself can scavenge HOCl, it was examined whether DTBP attenuated HOCl-induced endothelial dysfunction. Indeed, dietary DTBP preserved the vascular function of isolated rings to an extent comparable to that seen with probucol (FIG. 6A). In contrast, rings from rabbits supplemented with BP that unlike probucol and DTBP lacks the sulfur atoms, responded to HOCl indistinguishably from control (FIG. 6A). As with probucol, the protection seen with dietary DTBP was associated with an increase in tissue cGMP, whereas supplementation with BP was ineffective (FIG. 6B), although all three phenols accumulated to a comparable extent (FIG. 6C). DTBP also protected aortic rings from HOCl-induced dysfunction when added in vitro, to an extent comparable to that seen with the identical concentrations of probucol (FIG. 7).

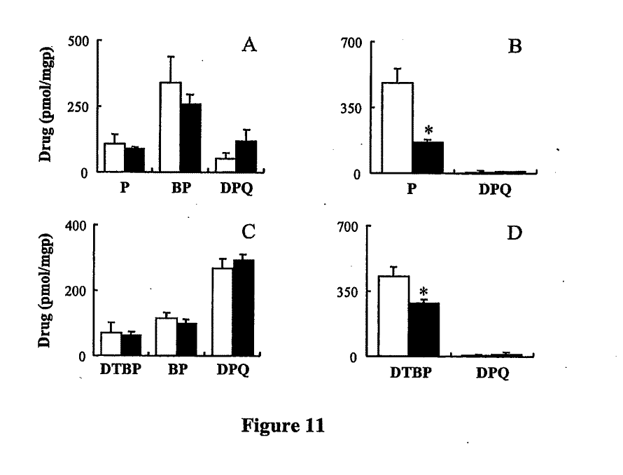

Finally, tissue samples from the vascular reactivity studies were assessed for their contents of probucol and DTBP as well as their respective oxidation products (FIG. 11). Rings from probucol-supplemented animals contained probucol, BP and DPQ, and treatment with HOCl tended to decrease tissue probucol and BP, and to increase DPQ (FIG. 11A), although this did not reach statistical significance; products other than BP and DPQ were not detected (not shown). In aortic rings to which probucol was added in vitro, subsequent treatment with HOCl significantly decreased tissue probucol without substantial accumulation of DPQ; BP was not detected (FIG. 11B). Results comparable to those with probucol were obtained with rings from animals supplemented with DTBP (FIG. 11C) and rings to which DTBP was added in vitro prior to HOCl exposure (FIG. 11D).

### Discussion

Vascular endothelial cells overlying atherosclerotic lesions contain myeloperoxidase and proteins modified by its principle product HOCl, and blood vessels exposed to HOCl exhibit a defect in endothelium-derived NO bioactivity manifested as impaired endothelium-dependent arterial relaxation. These results show that dietary or exogenously added probucol attenuated this vascular dysfunction induced by HOCl, and that HOCl oxidized probucol to DPQ with intermittent formation of DTBP. Similarly, dietary or exogenously added DTBP accumulated in the vessel wall, protected the vessel against HOCl-induced endothelial dysfunction, and scavenged HOCl to an extent comparable to probucol. Together, these results suggest that probucol is a pro-drug with DTBP the active metabolite that can protect against HOCl-mediated endothelial dysfunction.

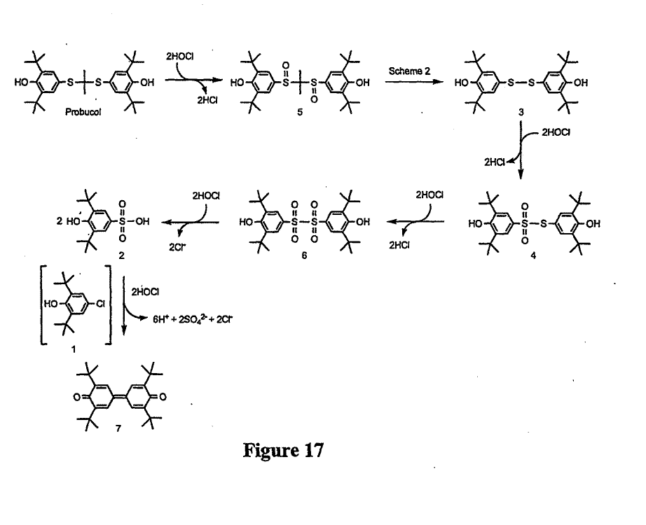

Impaired endothelial function predicts the occurrence of vascular events and NO bioavailability is attenuated by irreversible chemical modification and/or decreased catalytic activity of eNOS. Oxidative reactions are increasingly implied in these processes. This study shows that probucol and DTBP scavenge HOCl and that DTBP is an intermediate during HOCl-mediated oxidation of probucol. Probucol and DTBP also react with other oxidants. Using Cu²⁺-ions, Barnhart et al. (Journal of Lipid Research 1989, 30:1703-1710) described BP and DPQ as oxidation products of probucol, with a spiroquinone proposed as intermediate. The spiroquinone was not detected, independent of whether Cu²⁺, HOCl or other oxidants were employed, including PbO₂ used by Barnhart et al. (Journal of Lipid Research 1989, 30:1703-1710) to produce spiroquinone standard, and whether the HPLC conditions described here or by Barnhart et al. (Journal of Lipid Research 1989, 30:1703-1710) were used for detection of products. Notwithstanding this, DTBP was detected consistently and to an extent proportional to the yield of DPQ, independent of whether 2e- or 1e-oxidants were used (not shown). Overall, the present data suggest that oxidation of the sulfur atoms to the disulfoxide 5 is the first step in HOCl-mediated conversion of probucol to DTBP 3 (FIG. 17). This is distinct from the oxidation of probucol's phenolic group.

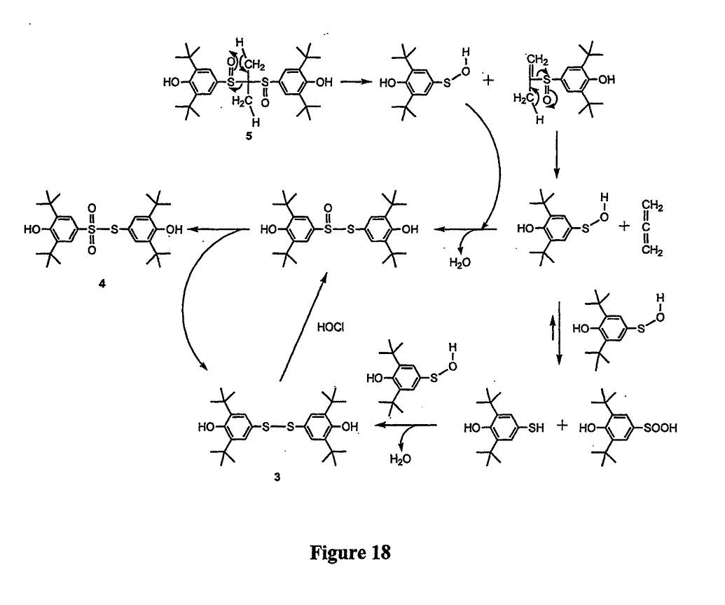

The precise mechanism for the rearrangement of 5 to 3 remains to be elucidated. Whilst not intending to be bound or limited to a specific mechanism, one chemically feasible pathway may be via a non-radical mechanism (FIG. 18). In this scheme, DTBP is formed via disproportionation of two molecules of thiosulfinate, or via coupling of the thiophenol and phenylsulfonic acid. The present results suggest that DTBP is further oxidized by HOCl to yield the thiosulfonate 4 that oxidizes to the disulfone 6, which becomes hydrolysed to the corresponding sulfonic acid 2. The acid product 2 is then chlorinated by another molecule of HOCl to yield the chlorophenol 1 and 2H⁺/SO₄²⁻ as by-product that was detected in HOCl-mediated oxidations of probucol and DTBP. Finally, the chlorophenol intermediate is converted to DQ. Irrespective of the precise mechanism of its formation, DQ is degraded in the presence of excess HOCl.

Unlike probucol and DTPB, BP failed to protect vessels from HOCl-induced endothelial dysfunction (FIG. 6). This indicates that radical scavenging by the phenolic moiety alone cannot be responsible for the protection seen with probucol and DTBP, a conclusion also in line with the observation that vitamin E, another phenolic antioxidant, fails to protect against HOCl-mediated endothelial dysfunction. A comparison of the efficacy of probucol, DTBP and BP points to the importance of the sulfur atoms for protection against HOCl-mediated endothelial dysfunction.

Close examinations of the data indicate that the observed inhibition of HOCl-mediated endothelial dysfunction by probucol and DTBP is not likely due to direct scavenging of HOCl. First, the rate constants for the reaction of HOCl with probucol and DTBP are orders of magnitude lower than those for reaction of HOCl with several biological targets, such as heme, ascorbate and amino acids. Based on kinetic arguments therefore, direct reaction of HOCl with probucol or DTBP present in the vessel wall is not favoured. Consistent with this argument, probucol- or DTBP-containing vessels exposed to HOCl did not contain measurable oxidation products despite a clear consumption of the respective phenolic compounds (FIG. 11).

### Example 5

### DTBP, but not BP, has Anti-Atherosclerotic Activity

This example illustrates that the phenol moiety of probucol is not sufficient for anti-atherosclerotic activity, and that instead the sulfur moieties are required. In addition, this example illustrates that DTBP at 1/50^{th} of the dose of probucol has anti-atherosclerotic protection comparable to that of probucol yet, unlike probucol, does not lower HDL-cholesterol.

### Materials and Methods

ApoE−/− mouse model. Four groups of male apoE−/− mice (8-10 weeks, Animal Resources Centre, Perth, Australia) were fed a high fat diet based on Harlan Teklad diet TD88137±1% (wt/wt) probucol (96% purity, a gift from AstraZeneca, Sweden), 0.02% DTBP, or 0.02% BP (Polysciences, Warrington, Pa.) for 5 months. Tissue harvesting and analyses were done as described in Examples 1 and 3, using 15 and 10 mice of each group for biochemical and histological analyses, respectively.
Histology and immunohistochemistry. Aortic lesion assessment was carried out at four sites (sinus, arch, thoracic and abdominal aorta) as described in Example 1. Immediately adjacent sections were employed for immunohistochemistry, using Mac-3 (macrophages, dilution 1:200, DAKO), PCNA (cell proliferation, dilution 1:500, DAKO) and anti-rat HO-1 monoclonal antibody (dilution 1:50, Santa Cruz) with avidin-biotin-horseradish peroxidase for signal detection (Vectorstain Elite ABC Kit, Vector Laboratories). Apoptosis was assessed using the TUNEL assay kit (Roche) according to the manufacture's instructions. Digital images were taken for quantitative morphometric analysis. Mac-3⁺ areas were determined using Adobe Photoshop V6.0 by tracing. Total cell profiles, TUNEL⁺ and PCNA⁺ cells were counted manually at high magnification (40× objective). A single operator using coded samples performed all analyses blinded.
Heme oxygenase. Heme oxygenase activity was determined in microsomes prepared from homogenized aortic tissue and assessed by HPLC as described in Circulation 2004, 110: 1855-1860.
Statistics. All data are expressed as mean±SEM. One-way ANOVA and the student-Newman-Keul's test were used to evaluate differences between groups, with P<0.05 considered significant.

### Results

Example 1 shows that probucol affects atherosclerosis in apoE−/− mice in a site-specific manner, enhancing lesion size at the aortic root and strongly inhibiting disease at the descending aorta. It also shows that inhibition of atherosclerosis in the thoracic and abdominal aorta by probucol is associated with oxidative metabolism of probucol to BP. Example 4 shows that that DTBP is an intermediate in the oxidative conversion of probucol to BP, and that DTBP has biological protective activity in that it inhibits HOCl-mediated endothelial dysfunction. To test whether DTBP has also anti-atherosclerotic activity, the apoE−/− mouse model as described in Example 1 was used. To determine the structural requirements underlying the anti-atherosclerotic activity of probucol, the effect of 1% (w/w) probucol was compared with that of 0.02% DTBP and 0.02% BP on atherosclerosis in apoE−/− mice fed a high fat diet for 5 months. The drug dosages chosen resulted in total aortic drug concentrations of 21.8±8.4, 7.1±1.4 and 92.8±18 nmol/mg protein for probucol, DTBP and PB, respectively (n10, p<0.05 for DTBP versus probucol and BP, and for BP versus probucol). Given that DTBP was used at only 1/50^{th} of probucol's dose, these results show that DTBP has clearly superior bioavailability compared to probucol.

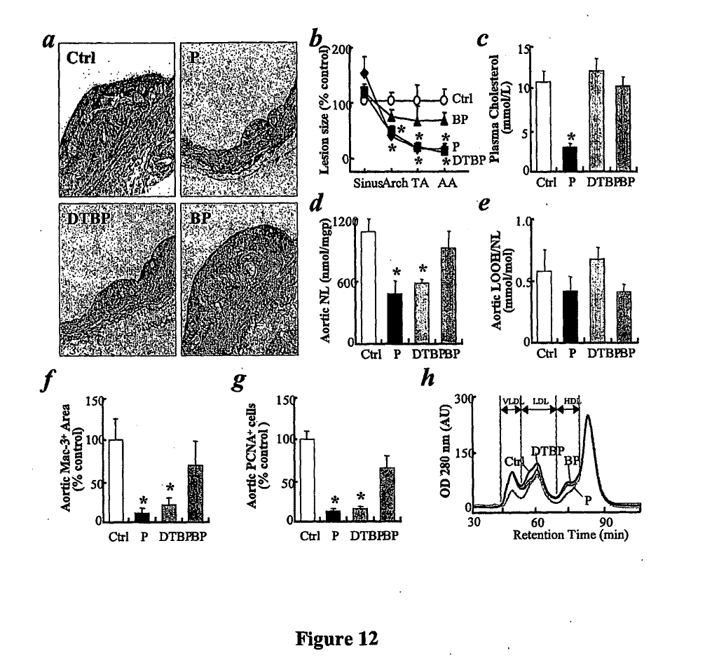

As expected from the results shown in Example 1, probucol affected lesion formation in the established site-specific manner (FIG. 12b). Like probucol, DTBP also significantly inhibited atherosclerosis at the arch and descending aorta (FIG. 12b). Unlike probucol however, DTBP did not increase medium lesion size at the aortic root (FIG. 12b) or affect plasma cholesterol (FIG. 12c) and HDL concentration (FIG. 12h). In contrast to probucol and DTBP, BP failed to both, inhibit atherosclerosis (FIG. 12b) and decrease plasma cholesterol (FIG. 12c). Consistent with the morphometric lesion assessment, probucol and DTBP, but not BP, decreased the aortic content of neutral lipids (cholesterylesters and triglycerides) (FIG. 12d), independent of the content of oxidized lipids in the affected vessel wall (FIG. 12e). Rather, inhibition of atherosclerosis by probucol and DTBP was associated with a significant decrease in both macrophages lesion area (FIGS. 12a and f) and proliferating cells (FIG. 12g), whereas BP failed to affect these parameters. The results indicate that DTBP is a potential anti-atherogenic compound that may not share some of the undesirable side effects of probucol, such as the lowering of HDL-cholesterol. As the drug concentration in the aorta of DTBP-treated animals was significantly lower than that in probucol-treated animals, the results also show that DTBP has greater anti-atherosclerotic activity than probucol, separately from its comparatively higher bioavailability than probucol. Finally, the lack of significant protective activity of BP, despite aortic accumulation of the drug at concentrations that exceeded those of probucol- and DTBP-treated animals, unambiguously shows that the phenol moiety of probucol is not sufficient for in vivo protective activity. Instead, the results document that the sulfur moieties of probucol, that are present in DTBP, but not BP, are required for anti-atherosclerotic activity.

### Example 6

### Probucol and DTBP, but not BP, Inhibit Intimal Thickening Following Injury

This example illustrates that the phenol moiety of probucol is not sufficient for inhibition of injury-induced intimal thickening, and that instead the sulfur moieties are required.

### Materials and Methods

Materials and methods were as described in Example 5, with the following additions.

Rabbit aortic balloon-injury model. Four groups of male New Zealand White rabbits (1.8-2.2 kg, Merunga Farm, Coffs Harbour, Australia), matched for body weight and baseline plasma cholesterol, were fed 100 g per day of normal chow±1% probucol, 0.02% DTBP, or 0.02% BP (wt/wt) for up to nine weeks. Aortic balloon-injury (ABI) was carried out at the end of week three, resulting in complete endothelial denudation. Harvesting of aortas was done after a further 6 weeks (n=6 per group). Aortic lesion assessment was carried out at the 3^{rd} pair of lumber arteries in NZW rabbits as described in Circulation 2003, 107:2031-2036.
Statistics. Data are expressed as mean±SEM. One-way ANOVA and the student-Newman-Keul's test were used to evaluate differences between groups, with P<0.05 considered significant.

### Results

As the results in Example 5 showed that DTBP has anti-atherosclerotic activity, the structure-function study was repeated in a rabbit model of intimal hyperplasia in response to injury. To control for probucol's cholesterol-lowering effect, the animals were matched for baseline plasma cholesterol, and fed them a limited amount (100 g/day) of standard diet±the respective drug. Results from a pilot study showed that treatment of rabbits (n=6 per treatment) for 9 weeks with 1% (w/w) probucol, 0.02% DTBP or 0.02% BP resulted in 663±114, 235±64 and 614±258 pmol total drug per mg protein in the vessel wall for probucol, DTBP and BP groups, respectively (p>0.05 for all comparisons). Therefore, similar to the situation in apoE−/−, DTBP showed greater bioavailability than probucol in an animal model employing standard (rather than high fat) diet.

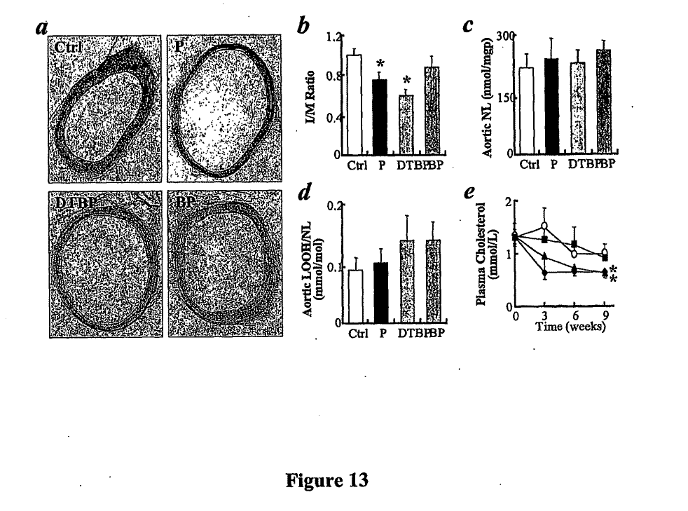

Also similar to the situation in apoE−/− mice, DTBP, but not BP, was as protective as probucol (FIG. 13a), significantly decreasing the intima-to-media ratio (FIG. 13b), without altering the vessel content of non-oxidized (FIG. 13c) and oxidized lipid (FIG. 13d). Compared to control, none of the drugs used affected vessel remodelling, as assessed by the length of the external elastic lamina and lumen area (not shown). Probucol (and BP) significantly reduced plasma total cholesterol concentration (FIG. 13e), whereas DTBP was without effect, consistent with the results in apoE−/− that DTBP does not lower HDL cholesterol. Comparison of the relative aortic drug concentrations and in vivo protection seen with probucol and DTBP, these results further support the notion that on a molar base, DTBP is more active than probucol. The fact that, like in the apoE−/− mouse model, BP also lacked protective activity in this model of intimal hyperplasia, further documents that the sulfur atoms, rather than the phenol moieties are required for protection against cardiovascular disease.

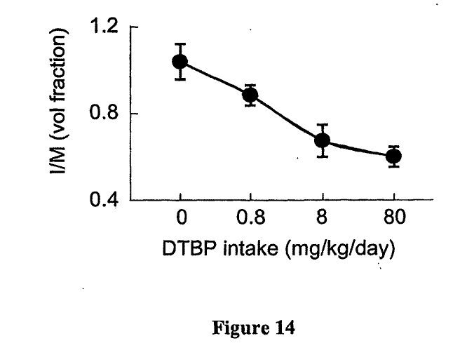

The results in FIG. 14 show that DTBP provided protection against balloon injury-induced intimal hyperplasia in a concentration-dependent manner.

### Example 7

### In Vivo Protection by DTBP Against Intimal Thickening Following Injury is Associated with the Promotion of Re-Endothelialization and Inhibition of Proliferation of Vascular Smooth Muscle Cells Via Induction of Heme Oxygenase

This example illustrates that the protective activity of DTBP relates to the ability of the disulfide to both accelerate the re-growth of a functional endothelium, and to induce heme oxygenase-1 in vascular smooth muscle cells that itself induces apoptosis and causes subsequent inhibition of proliferation, resulting in inhibition of intimal thickening.

### Materials and Methods

Materials and methods were as described in Example 6, except that for some experiments, aortas were also harvested 4 days (n=8 per group) after balloon injury. Sections immediately adjacent sections to those used for lesion assessment were employed for immunohistochemistry, using CD31 (endothelium, dilution 1:50, DAKO), Mac-3 (macrophages, dilution 1:200, DAKO), PCNA (cell proliferation, dilution 1:500, DAKO) and anti-rat HO-1 monoclonal antibody (dilution 1:50, Santa Cruz) with avidin-biotin-horseradish peroxidase for signal detection (Vectorstain Elite ABC Kit, Vector Laboratories). Apoptosis was assessed using the TUNEL assay kit (Roche) according to the manufacture's instructions. Digital images were taken for quantitative morphometric analysis. Intimal, medial and Mac-3⁺ areas were determined using Adobe Photoshop V6.0 by tracing. Re-endothelialization was determined in longitudinal sections as the distance of CD31⁺ cells from the branch orifice using Scion Image Software (Scion, ML, USA). Total cell profiles, TUNEL⁺ and PCNA⁺ cells were counted manually at high magnification (40× objective).

Vascular reactivity. Segments (3 mm) of the abdominal aorta at the 2^{nd} pair of lumber arteries from rabbits were used for isometric tension experiments, and segments extending proximally were analyzed for cGMP content as an index of NO synthase activity, as described in Circulation 2003, 107:2031-2036.
Heme oxygenase. RNA was isolated with TRIzol (Invitrogen) from RNAlater (Ambion) treated frozen tissues. cDNA was prepared using the Superscript III first strand synthesis kit using oligo (dT) primers (Invitrogen). Real-time PCR was performed on ABI PRISM 7700 Sequence Detection System using the SYBR Green PCR Master Mix (Applied Biosystems, Foster City, Calif.). Hydroxymethylbilane synthase (HMBS) was employed to normalize RNA quantity, using the following PCR primers: HMBS forward, 5′-GAGTGATTCGCGTGGGTACC-3′; HMBS reverse, 5′-GGCTCCGATGGTGAAGCC-3′; HO-1 forward, 5′-TGGAGCTGGACATGGCCTTC-3′; HO-1 reverse, 5′-TCTGGGCGATCTTCTTAAGG-3′. The amount of HO-1 mRNA was determined relative to HMBS mRNA using the comparative C_{T} method described in the ABI 7700 Sequence Detector User Bulletin 2. PCR products were verified by sequence analysis. Heme oxygenase activity was determined in microsomes prepared from homogenized aortic tissue and assessed by HPLC as described in Free Radicals in Biology & Medicine 1998, 24:959-971.
Statistics. All data are expressed as mean±SEM. One-way ANOVA and the student-Newman-Keul's test were used to evaluate differences between groups, while acetylcholine and sodium nitroprusside dose responses curves were compared by two-way ANOVA for repeated measures, with P<0.05 considered significant.

### Results

### DTBP Promotes Functional Re-Endothelialization

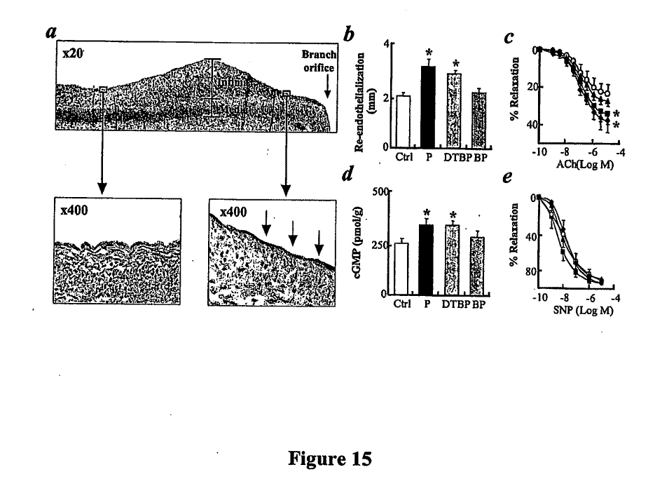

Re-endothelialization is a key repair process in response to arterial injury that is promoted by probucol. It was then assessed whether DTBP similarly promoted endothelial regeneration using longitudinal sections stained for the endothelial marker CD31. Six weeks after injury, the denuded aortic surface close to branch orifices was covered by endothelium that extended from arterial side-branches (FIG. 15a). Compared to control, DTBP and probucol, but not BP, significantly enhanced the regeneration of endothelium (FIG. 15b), and they significantly decreased the intima-to-media ratio determined at CD31-positive sites (not shown). Functional studies employing aortic rings taken from these sites where re-endothelialization was enhanced, showed that compared to control, all three drugs enhanced the norepinephrine-induced increase in vessel tone by ˜25% (not shown). In addition, DTBP and probucol, but not BP, enhanced endothelium-dependent relaxation (FIG. 15c) and tissue content of cGMP in response to acetylcholine (FIG. 15d), whereas the compounds had no effect on endothelium-independent relaxation induced by sodium nitroprusside (FIG. 15e). Thus, DTBP but not BP promoted the regeneration of functional endothelium, similar to probucol.

### DTBP Induces Heme Oxygenase-1 and Suppresses Neointimal Development

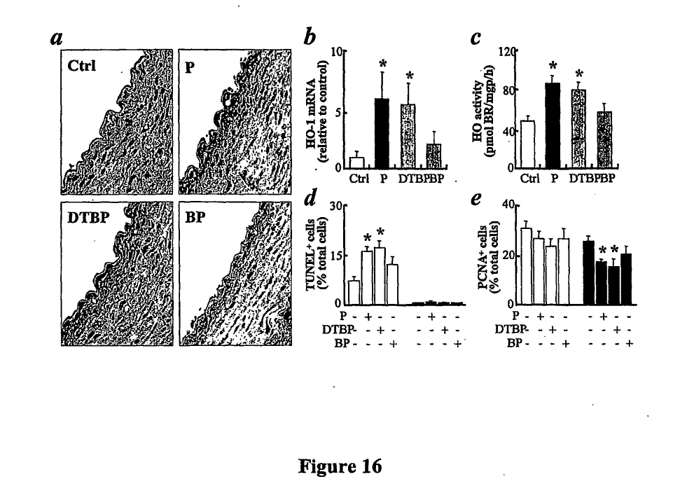

Increasing evidence points to a key role for the induction of HO-1 in the control of intimal hyperplasia, including that caused by probucol, as has been described recently by Deng et al. (Circulation 2004, 110:1855-1860). Immunohistochemical analyses of rabbit aortas as early as 4 days after balloon injury, showed that, DTBP and probucol, but not BP, induced HO-1 expression in the media close to the luminal side in damaged (FIG. 16a), but not undamaged aortas (not shown). Consistent with this, enhanced HO-1 expression was associated with significantly increased tissue levels of HO-1 mRNA (FIG. 16b) and heme oxygenase activity (FIG. 16c). As induction of HO-1 activity in vascular smooth muscle cells is linked to enhanced apoptosis that subsequently results in decreased proliferation, the effect of the three compounds on the number of vascular cells positive for TUNEL (a measure of apoptosis) and PCNA (cell proliferation) was examined. Probucol and DTBP, not BP, were observed to significantly enhanced apoptosis early, i.e., at day 4, but not at day 42, after aortic balloon injury (FIG. 16d), and this was associated with a significant decrease in cell proliferation late, i.e., at day 42 but not day 4 after ABI (FIG. 16e). Immunostaining for HO-1 was not longer detected in damaged vessels at day 42 (not shown). These results may imply a link between HO-1 induction and inhibition of intimal hyperplasia by DTBP, and may suggest that HO-1 is a target for the protective activity of the disulfide.

### Example 8

### Blocking Heme Oxygenase Activity Prevents the Ability of Probucol and DTBP to Promote Re-Endothelialization, Inhibit the Proliferation of Vascular Smooth Muscle Cells, and Protect Against Intimal Thickening Following Injury

This example provides in vivo evidence that heme oxygenase(s) is/are a target for probucol and DTBP, and that the protection observed with probucol and DTBP is dependent on the ability of the compounds to promote re-endothelialization and to inhibit smooth muscle cell proliferation.

### Materials and Methods

Materials and methods were as described in Examples 6 and 7, with the following addition.

Inhibition of heme oxygenase activity. Three groups of male New Zealand White rabbits (n=6 per group) that were fed 100 g per day of normal chow±1% probucol or 0.02% DTBP (wt/wt) received intraperitoneal injection of tin protoporphyrin (SnPP, Frontier Scientific, 7.5 mg/kg) every other day as described in American Journal of Physiology, Heart and Circulatory Physiology 2000; 278:H623-H632 for the entire nine week duration of the experiment, with balloon injury at the end of week three and lesion assessment after a further six weeks.

### Results

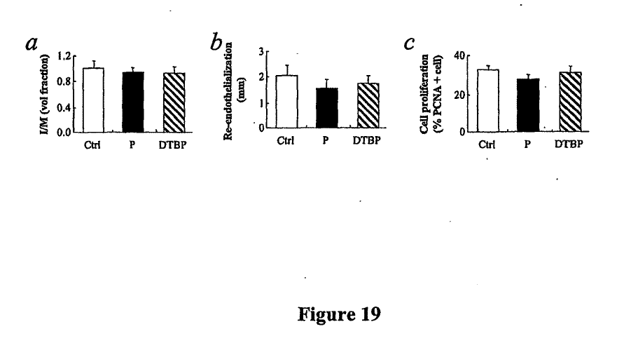

As the results in Example 7 showed that inhibition of intimal thickening by probucol and DTBP is associated with the promotion of re-endothelialization and inhibition of proliferation via induction of heme oxygenase-1 (HO-1), the requirement for heme oxygenase induction for the in vivo protective activities of probucol and DTBP was tested. For this, animal received tin protoporphyrin to inhibit heme oxygenase activity in addition to receiving normal chow without (control, ctrl), or with probucol (P), or DTBP for 9 weeks. Blocking heme oxygenase activity via administration of tin protoporphyrin completely prevented the ability of probucol and DTBP to inhibit intimal thickening in response to arterial balloon injury (FIG. 19a). At the same time, administration of tin protoporphyrin also completely prevented the ability of probucol and DTBP to promote re-endothelialization (FIG. 19b) and to inhibit vascular smooth muscle cell proliferation (FIG. 19c).

The results in FIG. 19 show induction of heme oxygenase is required for the in vivo protective activity of probucol and DTBP, thereby identifying heme oxygenase(s) as target(s) for these compounds. The results also show that both promotion of re-endothelialization and inhibition of proliferation of vascular smooth muscle cells represent biological processes through which probucol and DTBP inhibit intimal thickening after balloon injury.

### Example 9

### ‘Classic’ Phenolic Antioxidants, Such as the Radical Scavenger Vitamin E, Fail to Induce Heme Oxygenase, do not Promote Re-Endothelialization, and Also Fail to Protect Against Intimal Thickening Following Injury

This example contrasts the protective actions of probucol from those of ‘classic’ antioxidants, exemplified by the phenolic radical scavenger α-tocopherol, i.e., biologically the most active form of vitamin E.

### Materials and Methods

Materials and methods were principally as described in Examples 6 and 7. Vitamin E (α-tocopherol for cellular studies, α-tocopheryl acetate for in vivo studies) was obtained from Sigma (St. Louis, Mo.). Heme oxygenase-1 mRNA was assessed by real time RT-PCR in rabbit aortic smooth muscle cells cultured for 24 hours in the presence of vehicle (control), probucol (50 μM) or α-tocopherol (50 μM) as described in (Circulation 2004; 110:1855-1860). Re-endothelialization was assessed by Evans blue staining (Circulation 2003; 107:2031-2036) 3 weeks after injury. Intima-to-media ratio of vessels from control rabbits and animals treated with probucol or α-tocopheryl acetate (n=6 per group) was also determined 3 weeks after aortic balloon injury, using 5 serial sections per aortic segment, 100 μm apart.

### Statistics

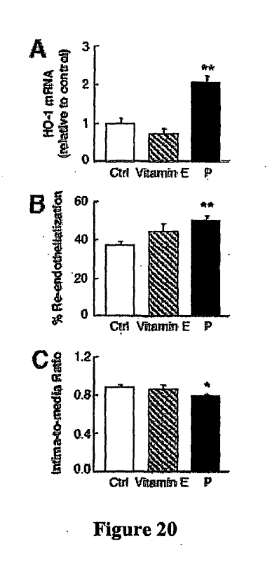

Data are expressed as mean±SEM. One-way ANOVA and the student-Newman-Keul's test were used to evaluate differences between groups. Results in FIG. 20A show mean±SEM of a triplicate experiment performed twice with similar results obtained in both experiments.

### Results

Unlike probucol, vitamin E failed to induce HO-1 in vascular smooth muscle cells in vitro (FIG. 20A). This was reflected by a lack of ability of the vitamin to promote re-endothelialization (FIG. 20B) and to inhibit intimal hyperplasia in vivo (FIG. 20C). These results show that vitamin E does not share the protective activities identified for probucol.

### Discussion

The oxidative modification hypothesis of atherosclerosis has been challenged recently by the failure of the ‘classic’ antioxidant vitamin E, either alone or in combination with vitamin C, selenium or β-carotene, to reduce disease progression and clinical events in patients at risk of or with established atherosclerosis (Physiological Reviews 2004; 84:1381-1478). Furthermore, a previous study (New England Journal of Medicine 1997; 337:365-372) reported co-administration of vitamin E plus vitamin C and β-carotene to block the ability of probucol to inhibit restenosis in human subjects undergoing balloon angioplasty. The authors of New England Journal of Medicine 1997; 337:365-372 did not explain this adverse effect of vitamin E plus vitamin C and β-carotene on the protective action of probucol. The results disclosed in Examples 6-9 together with recent reports for the first time provide a rationale for why ‘classic’ phenolic antioxidants fail to prevent atherosclerotic vascular disease. Thus, administration of vitamin E blocks rather than induces heme oxygenase-1 in vivo (Free Radicals in Biology and Medicine 2002; 32:1293-1303; Free Radical Research 2002; 36:633-639; Journal of Hepatology 2004; 41:815-822), whereas induction of heme oxygenase-1 is key to the protection observed with probucol and DTBP.

The results help explain why phenolic antioxidants like vitamin E have failed to protect against cardiovascular disease (Physiological Reviews 2004; 84:1381-1478). The observation that DTBP, but not BP, inhibited disease in the models used here (Examples 6-9) suggests that the sulphur rather than the phenol moieties are important for protection against atherosclerotic vascular disease by redox-active compounds. This notion is consistent with the finding that in the rabbit balloon injury model, vitamin E failed to inhibit intimal hyperplasia, and it did not promote re-endothelialization or induce HO-1 in smooth muscle cells (FIG. 20). Sulphur atoms commonly engage in 2e-oxidation reactions, against which phenolic radical (i.e., 1e-oxidant) scavengers like BP and vitamin E offer little protection (Physiological Reviews 2004; 84:1381-1478). This implies that 2e-redox reactions may be more important than radical reactions in the pathogenesis of atherosclerotic disease. Indeed, 2e-oxidant-mediated oxidation of cysteine residues in the thiolate form is increasingly linked to the regulation of key enzymes (e.g., thioredoxin, Ras GTPases, tyrosine kinases, phosphatases and transcription factors) involved in processes central to atherogenesis, such as cell proliferation (Science 1995; 270:296-299), endothelial function (The Journal of Clinical Investigations 2002; 109:817-826) and cell signaling (American Journal of Physiology, Cellular Physiology 2004; 287:C246-C256). Consistent with this notion, Example 4 shows that the oxidation of probucol's sulphur atoms by 2e-oxidants relates to the extent to which this antioxidant provides vascular protection, while Examples 5-7 show that probucol's sulphur atoms are required for protection against atherosclerotic vascular disease.

### Example 10

### Synthesis of DTBP-s and STBP

### A. Synthesis of 4-(2,6-di-t-butyl-4-((3,5-di-t-butyl-4-hydroxyphenyl)disulfanyl)phenoxy)-4-oxobutanoic acid (DTBP-s)

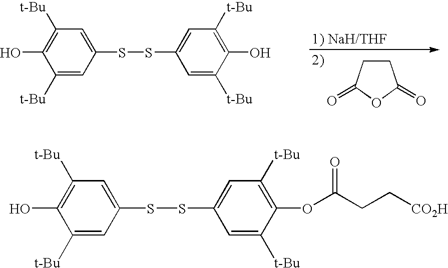

10 g (21 mmol) of 4,4′-disulfanediylbis(2,6-di-t-butylphenol) in 50 mL of dry THF was added slowly using an addition funnel to a stirred suspension of 2.0 g (84 mmol) of sodium hydride in 200 mL of dry THF under nitrogen and with cooling in an ice bath. During the addition, vigorous evolution of hydrogen gas was observed. The mixture was stirred for a further 15 minutes and 8.40 g (84 mmol) of succinic anhydride was added in two portions. The mixture was allowed to slowly warm up to room temperature and was stirred overnight. The next day, the mixture was cooled again to 0° C. and 5 ml of water was slowly added. THF was removed under reduced pressure and the dark residue was extracted with boiling hexane. The combined yellow extracts were evaporated and purified by flash silica gel chromatography (starting with hexane/ethyl acetate 6:2 and ending with 100% ethyl acetate) to give 3.00 g of the monoester (24.7%). [For the final purification, several batches were combined and re-crystallized three times from hexane/ether]. 4,4′-disulfanediylbis(2,6-di-tert-butylphenol) may be prepared by a literature procedure (T. Fujisawa, M. Yamamoto, G-I. Tsuchihashi Synthesis 1972, 624-5).

### B. Synthesis of 2,6-di-t-butyl-4-(3,5-di-t-butyl-4-hydroxyphenylselanylthio)phenol (STBP)

### 2,6-di-butyl-4-mercaptophenol

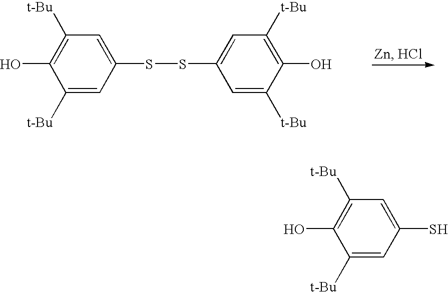

10 g (21 mmol) of 4,4′-disulfanediylbis(2,6-di-t-butylphenol) was dissolved in 100 mL of anhydrous ethanol, 8 g of zinc powder was added and the mixture was stirred and cooled in an ice bath. Concentrated HCl (18 mL) was added dropwise and the progress of the reduction was monitored by thin layer chromatography. After 30 minutes there was still some of the starting material left and more zinc (2 g) was added. After another 30 minutes the reaction was complete. The reaction mixture was diluted with water (300 mL) and extracted with hexane. The combined hexane extracts were dried with magnesium sulfate and evaporated, and the solid residue was crystallized from pentane to give 8.1 g (81%) of the product.

### 2,6-di-t-butyl-4-selenocyanatophenol

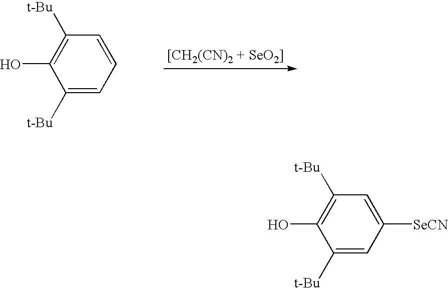

4.27 g of malonitrile (64 mmol) was dissolved in 80 mL of DMSO and 14.34 g of selenium dioxide (129 mmol) was added. The reaction mixture turned orange and eventually became dark. The reaction was stirred magnetically and monitored for gas evolution (attached rubber balloon). The gas started to evolve within 15 minutes and the reaction became warm to the touch. The reaction mixture was then briefly cooled with an ice bath and stirring was continued at room temperature. After 45 minutes the evolution of gas ceased and very little selenium dioxide remained. The stirring was continued for a further 15 minutes and 20.0 g of 2,6-di-t-butylphenol was added in one portion. After 1 hour and 40 minutes 400 mL of water was added and the stirring was continued for another 30 minutes. The product was filtered off, air-dried and crystallized from hexane to give 18.0 g (60% yield) of colorless crystals.

### 2,6-di-t-butyl-4-(3,5-di-1-butyl-4-hydroxyphenylselanylthio)phenol (STBP)

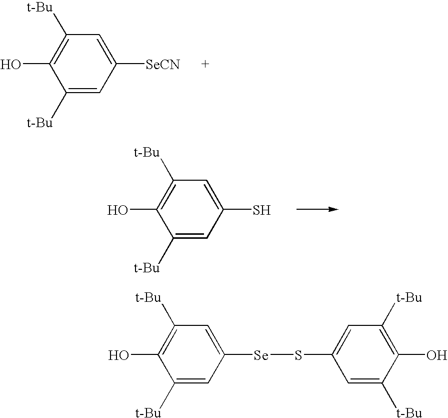

1 g of aluminum oxide (weakly acidic, Brockmann I) was added to a solution of 7.00 g (29.4 mmol) of 2,6-di-t-butyl-4-mercaptophenol in 150 mL of benzene. The mixture was stirred magnetically at room temperature. A gentle stream of argon was passed through the reaction mixture and a solution of 9.11 g (29.4 mmol) 2,6-di-t-butyl-4-selenocyanatophenol in 50 ml of benzene was added in portions over 15 minutes. The mixture was stirred for one hour and 15 minutes and then filtered and partially evaporated (ca. 40 mL left). 100 mL of pentane was added (some crystals started to form) and the solution was placed in a freezer overnight at −20° C. The next day, the crystalline product was filtered and rinsed with cold (−20° C.) pentane. 10.75 g of the product was obtained. Combined washings and mother liquor were partially evaporated, pentane was added to the residue and the crystallization was repeated to give 3.50 g of the second crop of the product. Total yield was 93%.

### Example 11

### Novel DTBP Analogues Protect Against Intimal Thickening Following Injury

This example establishes that novel analogues of DTBP provide protection against balloon injury-induced intimal hyperplasia.

### Materials and Methods

Materials and methods were as described in Examples 6 and 7, with the following addition. DTBP-s and STBP were synthesized as described in Example 10. A total of 48 New Zealand White rabbits (1.8-2.2 kg) were used for this example. Rabbits were fed normal chow (100 g/day) without (Ctrl, n=12) or with 0.02% DTBP (wt/wt, n=6), 0.02% STBP (wt/wt, n=7), 0.02% DTBP-s (wt/wt, n=11) or 0.1% DTBP-s (wt/wt, n=7) for 9 weeks. Aortic balloon-injury was carried out at the end of week three.

### Statistics

Data are expressed as mean±SEM. One-way ANOVA and the student-Newman-Keul's test were used to evaluate differences between groups. *P<0.01 versus Ctrl; #P<0.05 versus DTBP-s (0.02%).

### Results

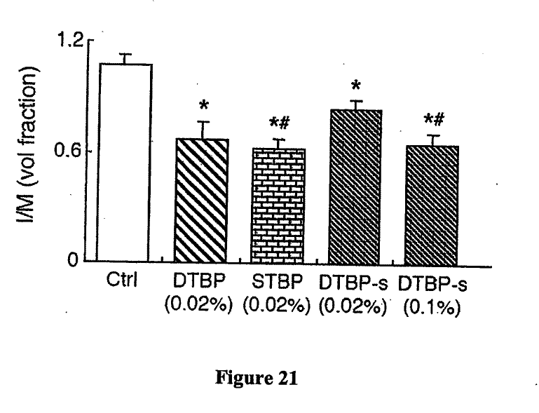

As shown in Example 6, DTBP at 0.02% (wt/wt) provided significant protection against balloon injury-induced intimal hyperplasia (FIG. 21). The seleno-analogue STBP (0.02%, wt/wt) protected to a comparable extent (FIG. 21). DTBP-s (0.02%, wt/wt) also provided significant protection, albeit less effectively as that seen with STBP (0.02%, to wt/wt) and DTBP-s (0.1%, wt/wt) (FIG. 21).

### Discussion

The results shown provide further evidence that the phenolic moiety of probucol and DTBP are not sufficient for in vivo protection against intimal hyperplasia. They also demonstrate that DTBP analogues that contain sulphur or selenium, i.e., moieties that readily engage in 2e-redox reactions, are a novel class of agents that provide protection against atherosclerotic vascular disease.

### Example 12

### Formulations

### Composition for Parenteral Administration

A pharmaceutical composition of the present invention for intramuscular injection could be prepared to contain 1-5 mL sterile buffered water, and 200-300 mg of a compound of Formula (I).

Similarly, a pharmaceutical composition for intravenous infusion may comprise 250 ml of sterile Ringer's solution, and 200-300 mg of a compound of Formula (I).

### Capsule Composition

A pharmaceutical composition of a compound of Formula (I) in the form of a capsule may be prepared by filling a standard two-piece hard gelatin capsule with 200-300 mg of a compound of Formula (I), in powdered form, 100 mg of lactose, 35 mg of talc and 10 mg of magnesium stearate.

### Injectable Parenteral Composition

A pharmaceutical composition of this invention in a form suitable for administration by injection may be prepared by mixing 1-5% by weight of a compound of Formula (I) in 10% by volume propylene glycol and water. The solution is sterilised by filtration.

### Composition for Inhalation Administration

For an aerosol container with a capacity of 20-30 ml: a mixture of 100-500 mg of a compound of Formula (I) with 0.5-0.8% by weight of a lubricating agent, such as polysorbate 85 or oleic acid, is dispersed in a propellant, such as freon, and put into an appropriate aerosol container for either intranasal or oral inhalation administration.

1. A compound of general Formula (I): 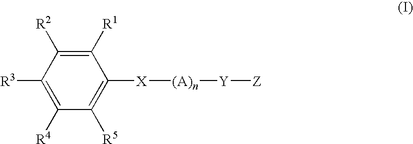
wherein
X is selected from S, Se, S(O) and S(O)₂;
Y is selected from S, Se, S(O) and S(O)₂;
wherein at least one of X and Y is Se;
A comprises one or more groups selected from optionally substituted C₁₋₆ alkylene, optionally substituted C₂₋₆ alkenylene; optionally substituted C₃₋₁₀ cycloalkylene; and optionally substituted arylene;
n is 0 or 1;
Z is selected from optionally substituted aryl and optionally substituted heteroaryl, optionally substituted alkyl, optionally substituted alkoxy, and NR¹³R¹⁴;
R¹, R², R³, R⁴, and R⁵ are the same or different and are independently selected from the group consisting of hydrogen, halogen, hydroxyl, thiol, —NR¹³R¹⁴, nitro, cyano, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted aryl, optionally substituted aryl(C₁₋₆ alkyl), optionally substituted (C₁₋₆ alkyl)aryl, optionally substituted heteroaryl, optionally substituted C₃₋₁₀ heterocycloalkyl, C(O)R¹¹, OR¹², SR¹², CH₂OR¹², CH₂NR¹³R¹⁴, C(O)OR¹² and C(O)NR¹³R¹⁴;
R¹¹ is selected from OH, C₁₋₆ alkyl, and C₂₋₆ alkenyl;
R¹² is selected from the group consisting of hydrogen, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted aryl, —C(O)(C₁₋₆)alkyl-CO₂R¹⁵, —C(O)(C₂₋₆)alkenyl-CO₂R¹⁵, and —C(O)NR¹³R¹⁴;
R¹³ and R¹⁴ may be the same or different and are individually selected from hydrogen, C₁₋₆ alkyl, C₂₋₆ alkenyl, C₂₋₆ alkynyl, C₃₋₆ cycloalkyl, C₃₋₆ heterocycloalkyl, aryl, (C₁₋₆)alkylaryl, and heteroaryl; and
R¹⁵ is H or C₁₋₄ alkyl;
with the proviso that the compound of formula (I) is not diphenyl diselenide or 4,4′-diselenobis[(2,6-di-tert-butyl)phenol];
and salts thereof.

2. A compound according to claim 1, wherein n is 1.

3. A compound according to claim 1, wherein n is 0.

4. A compound according to claim 1, wherein Z is an optionally substituted aryl group.

5. A compound according to claim 4, wherein Z is optionally substituted phenyl.

6. A compound according to claim 1, wherein, R³ is selected from hydroxyl, O-malonate, O-succinate, O-glutarate, O-adipate, O-maleate and O-fumarate.

7. A compound of general Formula (Ia): 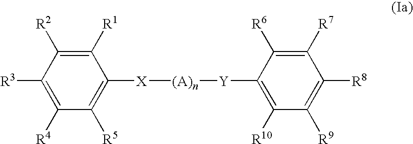
wherein
X is S or Se;
Y is S or Se;
wherein at least one of X and Y is Se;
A comprises one or more groups selected from optionally substituted C₁₋₆ alkylene, optionally substituted C₂₋₆ alkenylene; and optionally substituted C₃₋₁₀ cycloalkylene;
N is 0 or 1;
R¹, R², R³, R⁴, R⁵, R⁶, R⁷, R⁸, R⁹, and R¹⁰ may be the same or different and are independently selected from the group consisting of hydrogen, halogen, hydroxyl, thiol, —NR¹¹R¹², nitro, cyano, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted aryl, optionally substituted aryl(C₁₋₆ alkyl), optionally substituted (C₁₋₆ alkyl)aryl, optionally substituted heteroaryl, optionally substituted C₃₋₁₀ heterocycloalkyl, C(O)R¹¹, OR¹², CH₂OR¹², CH₂NR¹³R¹⁴, C(O)OR¹² and C(O)NR¹³R¹⁴;
R¹¹ is selected from OH, C₁₋₆ alkyl, and C₂₋₆ alkenyl;
R¹² is selected from the group consisting of hydrogen, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted aryl, —C(O)(C₁₋₆)alkyl-CO₂R¹⁵, —C(O)(C₂₋₆)alkenyl-CO₂R¹⁵, and —C(O)NR¹³R¹⁴;
R¹³ and R¹⁴ may be the same or different and are individually selected from hydrogen, C₁₋₆ alkyl, C₂₋₆ alkenyl, C₂₋₆ alkynyl, C₃₋₆ cycloalkyl, C₃₋₆ heterocycloalkyl, aryl, (C₁₋₆)alkylaryl, and heteroaryl;
R¹⁵ is H or C₁₋₄ alkyl;
with the proviso that the compound of formula (I) is not diphenyl diselenide or 4,4′-diselenobis[(2,6-di-tert-butyl)phenol];
and salts thereof.

8. A compound of general Formula (Ib): 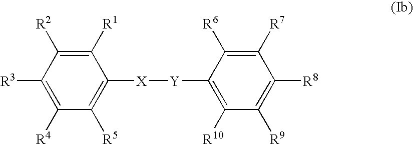
wherein
X is selected from S, Se, S(O) and S(O)₂;
Y is selected from S, Se, S(O) and S(O)₂;
wherein at least one of the X and Y is Se;
R¹, R², R³, R⁴, R⁵, R⁶, R⁷, R⁸, R⁹, and R¹⁰ are the same or different and are independently selected from the group consisting of hydrogen, halogen, hydroxyl, thiol, —NR¹¹R¹², nitro, cyano, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted aryl, optionally substituted aryl(C₁₋₆ alkyl), optionally substituted (C₁₋₆ alkyl) aryl, optionally substituted heteroaryl, optionally substituted C₃₋₁₀ heterocycloalkyl, C(O)R¹¹, OR¹², CH₂OR¹², CH₂NR¹³R¹⁴, C(O)OR¹² and C(O)NR¹³R¹⁴;
R¹¹ is selected from OH, C₁₋₆ alkyl, and C₂₋₆ alkenyl;
R¹² is selected from the group consisting of hydrogen, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted aryl, —C(O)(C₁₋₆)alkyl-CO₂R¹⁵, —C(O)(C₂₋₆) alkenyl-CO₂R¹⁵, and —C(O)NR¹³R¹⁴;
R¹³ and R¹⁴ are the same or different and are individually selected from hydrogen, C₁₋₆ alkyl, C₂₋₆ alkenyl, C₂₋₆ alkynyl, C₃₋₆ cycloalkyl, C₃₋₆ heterocycloalkyl, aryl, (C₁₋₆) alkylaryl, and heteroaryl;
R¹⁵ is H or C₁₋₄ alkyl;
with the proviso that the compound of formula (I) is not diphenyl diselenide or 4,4′-diselenobis [(2,6-di-tert-butyl)phenol];
and salts thereof.

9. A compound according to claim 1, wherein, X is S and Y is Se.

10. A compound according to claim 1, wherein X is Se and Y is S.

11. A compound according to claim 1, wherein X is Se and Y is Se.

12. A compound according to claim 1, wherein the optional substituents are independently selected from OH, SH, halogen, C₁₋₄ alkyl, C₂₋₄ alkenyl, O—(C₁₋₄ alkyl), S—(C₁₋₄ alkyl), cyano, amino, CO₂H and C(O)—O(C₁₋₆)alkyl.

13. A compound according to claim 7, wherein R³ and R⁸ are independently selected from hydroxyl, thiol, —NR¹³R¹⁴, cyano, C₁₋₆ alkyl, C₂₋₆ alkenyl, OR¹², C(O)OR¹² and C(O)NR¹³R¹⁴, wherein R¹², R¹³ and R¹⁴ are as defined in claim 1.

14. A compound according to claim 7, wherein R₃ and R₈ are independently selected from hydroxyl, O-malonate, O-succinate, O-glutarate, O-adipate, O-maleate and O-fumarate.

15. A compound of the general formula: 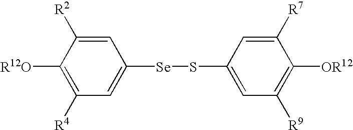
wherein
each R¹² is independently selected from hydrogen, C₁₋₁₀ alkyl and —C(O)(C₁₋₆) alkyl-CO₂R¹⁵;
R¹⁵ is selected from hydrogen and C₁₋₆ alkyl; and
R², R⁴, R⁷ and R⁹ are independently selected from methyl, ethyl, propyl, isopropyl, butyl, 1-methylpropyl, 2-methylbutyl, tert-butyl, pentyl, 2-methylpentyl, 3-methylpentyl and hexyl.

16. A compound selected from:

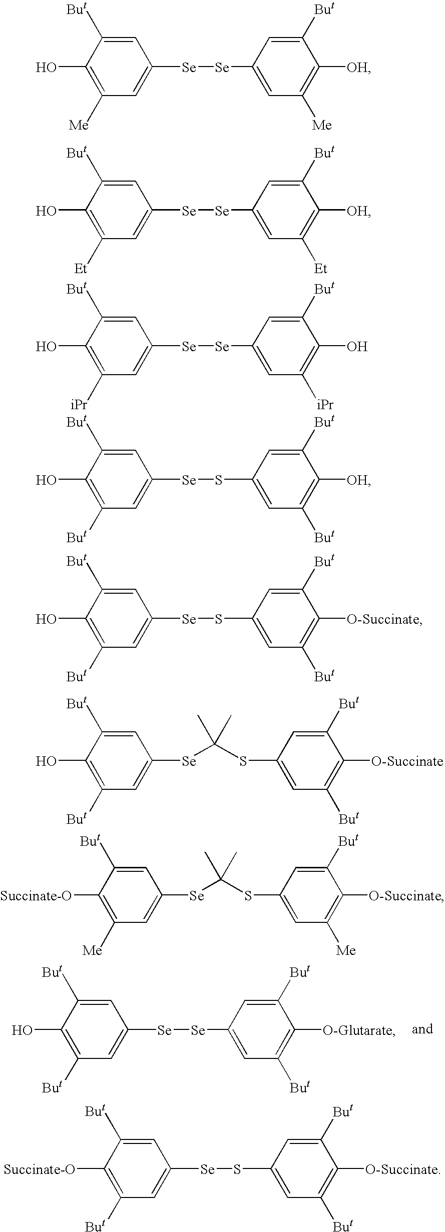

17. A compound of the general Formula: 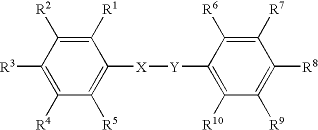
wherein
X is S;
Y is S;
R¹, R², R⁴, R⁵, R⁶, R⁷, R⁹, and R¹⁰ are the same or different and are independently selected from the group consisting of hydrogen, halogen, hydroxyl, thiol, —NR¹¹R¹², nitro, cyano, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted aryl, optionally substituted aryl(C₁₋₆ alkyl), optionally substituted (C₁₋₆ alkyl)aryl, optionally substituted heteroaryl, optionally substituted C₃₋₁₀ heterocycloalkyl, C(O)OR¹¹, OR¹², CH₂OR¹², CH₂NR¹³R¹⁴, C(O)OR¹² and C(O)NR¹³R¹⁴
one of R³ and R⁸ is selected from hydrogen, hydroxyl, thiol, —NR¹³R¹⁴, cyano, (C₁₋₆) alkyl, C₂₋₆ alkenyl, OR¹², C(O)OR¹² and C(O)NR¹³R¹⁴; and the other of R³ and R⁸ is selected from thiol, —NR¹³R¹⁴, cyano, (C₁₋₆) alkyl, C₂₋₆ alkenyl, OR¹², C(O)OR¹² and C(O)NR¹³R¹⁴; provided that when one of R³ and R⁸ is hydroxyl, the other of R³ and R⁸ is not OR¹² where R¹² is hydrogen;
R¹¹ is selected from OH, (C₁₋₆) alkyl, and C₂₋₆ alkenyl;
R¹² is selected from the group consisting of hydrogen, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted aryl, —C(O)(C₁₋₆)alkyl-CO₂R¹⁵, —C(O)(C₂₋₆)alkenyl-CO₂R¹⁵, and —C(O)NR¹³R¹⁴;
R¹³ and R¹⁴ are the same or different and are individually selected from hydrogen, C₁₋₆ alkyl, C₂₋₆ alkenyl, C₂₋₆ alkynyl, C₃₋₆ cycloalkyl, C₃₋₆ heterocycloalkyl, aryl, (C₁₋₆) alkylaryl, and heteroaryl;
R¹⁵ is H or C₁₋₄ alkyl;
provided that when R², R⁴, R⁵, R⁷, R⁹ and R¹⁰ are independently hydrogen, halogen, or alkyl, and one of R³ and R⁸ is hydrogen or alkyl and the other of R³ and R⁸ is alkyl, then R¹R⁶ are not both hydroxyl;
and salts thereof.

18. A compound according to claim 17, wherein one of R³ and R⁸ are independently selected from hydroxyl, O-malonate, O-succinate, O-glutarate, O-adipate, O-maleate and O-fumarate.

19. A compound selected from:

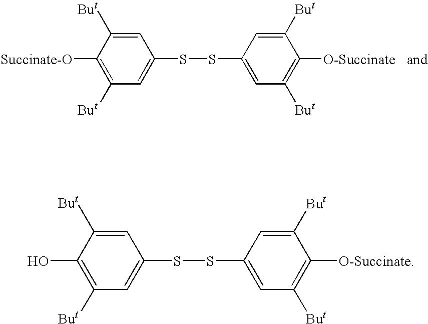

20. A pharmaceutical composition comprising at least one compound according to claim 1, together with pharmaceutically acceptable excipient, diluents and/or adjuvants.

21. A method of treating a cardiovascular disorder in a vertebrate, said method comprising administering to said vertebrate an effective amount of a compound according to claim 1 optionally together with a pharmaceutically acceptable excipient, diluent, and/or adjuvant.

22. The method according to claim 21, wherein said cardiovascular disorder is atherosclerosis.

23. The method according to claim 21, wherein said cardiovascular disorder is restenosis.

24. The method according to claim 21, wherein said method further comprises administering one or more agents selected from lipid-lowering drugs, antihypertensive drugs, beta blockers, diuretics, calcium channel blockers, and agents which promote induction of heme-oxygenase 1 (HO-1)

25. The method according to claim 24, wherein the lipid lowering drug is a statin.

26. The method according to claim 24, wherein the antihypertensive agent is an Angiotensin Converting Enzyme (ACE) inhibitor.

27. The method according to claim 21, wherein the vertebrate is human.

28-31. (canceled)
